(19) United States
(12) Patent Application Publication
(10) Pub. No.: US 2016/0348074 A1
(43) Pub. Date: Dec. 1, 2016

(54) METHODS AND COMPOSITIONS FOR TREATING CELLS FOR TRANSPLANT

(71) Applicant: Agenovir Corporation, South San Francisco, CA (US)

(72) Inventors: Stephen R. Quake, Stanford, CA (US); Jianbin Wang, South San Francisco, CA (US)

(21) Appl. No.: 15/166,934

(22) Filed: May 27, 2016

Related U.S. Application Data
(60) Provisional application No. 62/168,253, filed on May 29, 2015.

(51) Int. Cl. C12N 5/0789; A61K 35/28

(52) U.S. Cl. CPC C12N 5/0647; A61K 35/28; C12N 2510/00; C12N 2501/73

(57) ABSTRACT

The invention relates to methods for generating viral-free cells using nucleases for use in transplantation. The nucleases may be CRISPR/Cas9 complexes with guided RNA to target and inactivate viral genomes within cells. The nucleases degrade or destroy the viruses within the cells prior to transplantation.

### CROSS-REFERENCE TO RELATED APPLICATION

This application claims the benefit and priority of U.S. provisional patent application No. 62/168,253, filed May 29, 2015, the contents of which are incorporated by reference.

### FIELD OF THE INVENTION

The invention relates to hematopoietic stem cells transplant.

### BACKGROUND

A person with cancer who undergoes chemotherapy may then have to receive a bone marrow transplant, in which hematopoietic stem cells (HSCs) are delivered into the person's bone marrow. The transplant is necessary because the chemotherapy kills not only the cancerous cells, but also healthy cells that are necessary for normal immune function. The transplanted HSCs grow in the person's bone marrow and their progeny ultimately normalize the person's immune function. For the transplant to be successful, the donor's tissue type must match the person's. Due to that requirement, the pool of donors is usually limited to the person or their close relatives.

When the donated HCSs are infected by a virus, there can be severe consequences. In fact, a fourth of all bone marrow recipients die from a viral infection following transplantation. Donors may be wholly unaware that they carry a virus due to viral latency—the ability of a virus to lie dormant within a cell. The problem is compounded with viruses that themselves can cause cancer. For example, the Epstein-Barr virus (EBV), also called human herpesvirus 4 (HHV-4) is a virus that is associated with Hodgkin's lymphoma and Burkitt's lymphoma. If a latent virus is transmitted to a chemotherapy patient via a HSC transplant, it may subsequently reactivate and start producing progeny. Thus, a leukemia patient may beat one form of cancer, only to contract another form of cancer during treatment.

### SUMMARY

The invention provides methods for treating cells for viral infections. Cells may be treated ex vivo prior to delivery so that the person will not experience a viral infection from the cells following the transplant. A nuclease is used to target viral nucleic acid within the cells in vitro prior to delivery to the recipient. Where a cell is infected by a virus, the nuclease cleaves and thus interferes with the function of the viral nucleic acid, which prevents the virus from infecting the transplant recipient. Methods of the invention may be used to target latent viral infections within HSCs that have been obtained from a donor to ensure that those HSCs are virus-free prior to delivery to the recipient. Thus, stem cells that are donated for therapeutic treatments may be treated to eliminate viruses, even latent viruses such as the Epstein-Barr virus (EBV). The nuclease may be delivered as an active protein (or ribonucleoprotein, e.g., for Cas9), encoded in a nucleic acid such as a plasmid, or as messenger RNA. In some embodiments, a targetable nuclease is used to alter the genome of a virus, rendering it inactive. For example, stem cells may be transfected with an endonuclease such as Cas9 endonuclease and one or more guide RNAs that target the endonuclease to specific targets on the genome of the Epstein-Barr virus (EBV). In preferred embodiments, a ribonucleoprotein comprising a Cas9 nuclease and a guide RNA is delivered. Delivery preferably uses suitable materials or methods such as liposomes and/or electroporation. Once the virus is targeted and destroyed, the cells may then be used in therapeutic treatments without the risk of transmitting the virus to a transplant recipient. Thus, patients are able to benefit from bone marrow transplants without risk of a viral infection from the donor cells and certain associated risks of cancer.

In certain aspects, the invention provides methods for generating a viral-free cell. The methods may include obtaining a cell from a donor and delivering to the cell a nuclease that cleaves viral nucleic acid. The cell is then provided for transplantation to a patient.

It should be appreciated that any type of cell may be obtained from a donor. For example, exocrine secretory epithelial cells, hormone secreting cells, epithelial cells, sensory transducer cells, neuron cells, glial cells, lens cells, hepatocyte cells, adipocyte cells, lipocyte cells, kidney cells, liver cells, prostate gland cells, pancreatic cells, ameloblast epithelial cells, planum semilunatum epithelial cells, organ of Corti interdental epithelial cells, loose connective tissue fibroblasts, corneal fibroblasts (corneal keratocytes), tendon fibroblasts, bone marrow reticular tissue fibroblasts, pericytes, nucleus pulposus cells, odontoblast/odontocytes, chondrocytes, osteoprogenitor cells, hyalocytes, stellate cells, hepatic stellate cells, skeletal muscle cells, satellite cells, heart muscle cells, smooth muscle cells, myoepithelial cells, myoepithelial cells, erythrocytes, megakaryocytes, monocytes, connective tissue macrophages, epidermal Langerhans cells, osteoclasts, dendritic cells, microglial cells neutrophil granulocytes, eosinophil granulocytes, basophil granulocytes, hybridoma cells, mast cells, helper T cells, suppressor T cells, cytotoxic T cells, natural killer T cells, B cells, natural killer cells reticulocytes, somatic stem cells, embryonic stem cells, or hematopoietic stem cells may be used in methods of the invention. In some embodiments, the cell is infected with a virus and contains viral nucleic acid within the cell. The virus may be a herpes family virus. In some embodiments, the virus is in the latent stage in the cell.

It should also be appreciated that any type of nuclease may be used to cleave viral nucleic acid. The nuclease may be a zinc finger nuclease, a transcription activator-like effector nuclease, or a meganuclease. The nuclease may be a structure specific nuclease or a sequence specific nuclease. In some embodiments, the nuclease is a Cas9 endonuclease. The methods of the invention may further comprise cleaving the viral nucleic acid using the nuclease.

In some methods of the invention, the patient is a pre-determined person, such as a patient needing cells for transplantation. The patient may have a human leukocyte antigen (HLA) type that is matched to the donor. The cells may then be harvested from the donor; for example, from the donor's bone marrow or peripheral blood.

In some embodiments, the methods comprise delivering to the cell a guide RNA that targets the nuclease to a portion of the viral nucleic acid. In some embodiments, for example, a guide RNA targets the Cas9 endonuclease to a portion of the viral nucleic acid. In certain embodiments, the guide RNA is designed to have no perfect match in a human genome. The guide RNAs may target the nuclease to a regulatory element in the genome of the virus.

In some embodiments, the methods comprise delivering the nuclease to a plurality of cells from the donor. The plurality of cells is cultured, and cells where the nuclease successfully cleaves viral nucleic acid are selected. In some embodiments, a fluorescent marker is delivered with the nuclease, thus allowing cells that have cleaved viral nucleic acids to be selected. In some embodiments, the cells that are selected are then used in transplantation.

The nuclease may be delivered to the cell by a viral vector. The viral vector may be retrovirus, lentivirus, adenovirus, herpesvirus, poxvirus, alphavirus, vaccinia virus or adeno-associated viruses. In some embodiments, the nuclease may be delivered by a plasmid, a nanoparticle, a cationic lipid, a cationic polymer, metallic nanoparticle, a nanorod, a liposome, a cell-penetrating peptide, a liposphere, and polyethyleneglycol (PEG).

Any suitable virus may be treated using methods of the invention, such as Adenovirus, Herpes simplex, type 1, Herpes simplex, type 2, Varicella-zoster virus, Epstein-Barr virus, Human cytomegalovirus, Human herpesvirus, type 8, Human papillomavirus, BK virus, JC virus, Smallpox, Hepatitis B virus, Human bocavirus, Parvovirus B19, Human astrovirus, Norwalk virus, coxsackievirus, hepatitis A virus, poliovirus, rhinovirus, Severe acute respiratory syndrome virus, Hepatitis C virus, yellow fever virus, dengue virus, West Nile virus, Rubella virus, Hepatitis E virus, Human immunodeficiency virus (HIV), Influenza virus, Guanarito virus, Junin virus, Lassa virus, Machupo virus, Sabiá virus, Crimean-Congo hemorrhagic fever virus, Ebola virus, Marburg virus, Measles virus, Mumps virus, Parainfluenza virus, Respiratory syncytial virus, Human metapneumovirus, Hendra virus, Nipah virus, Rabies virus, Hepatitis D, Rotavirus, Orbivirus, Coltivirus, or Banna virus

In some aspects, the invention provides a method for generating a viral-free cell for use in transplantation where the method comprises obtaining a cell from a donor and then delivering to the cell an antiviral endonuclease that specifically targets one or more portions of a virus genome within the cell. The antiviral endonuclease binds to and alters the viral genome. The cell may then be provided for transplantation. In some embodiments, the treated cell is a stem cell, such as a hematopoietic stem cell. In some embodiments, a guided sequence may be used to target the antiviral endonuclease to the viral genome.

In some embodiments, the methods of the invention may further comprise using the cells treated with the antiviral endonuclease for cell culturing, where a population of cells is grown from the treated cell. In some embodiments, a fluorescent marker is added to a treated cell for optical detection.

In some embodiments, the antiviral endonuclease is a CRISPR/Cas9 endonuclease. A guide RNA that specifically targets one or more portions of a genome of a virus within a cell of the transplant may be used. The CRISPR/Cas9 complex binds to and alters the viral genome. In other embodiments, the invention may make use of a CRISPR/Cas9 nuclease and guide RNA (gRNA) that together target and selectively edit or destroy viral genomic material. The CRISPR (clustered regularly interspaced short palindromic repeats) is an element of the bacterial immune system that protects bacteria from phage infection. The guide RNA localizes the CRISPR/Cas9 complex to a viral target sequence. Binding of the complex localizes the Cas9 endonuclease to the viral genomic target sequence causing breaks in the viral genome. In a preferred embodiment, the guide RNA is designed to target multiple sites on the viral genome in order to disrupt the viral nucleic acid and reduce the chance that it will functionally recombine.

The presented methods allow for viral genome destruction, which results in the inability of the virus to proliferate, with no observed cytotoxicity to the cells. Aspects of the invention provide for designing a CRISPR/gRNA/Cas9 complex to selectively target viral genomic material (DNA or RNA), delivering the CRISPR/gRNA/Cas9 complex to a cell containing the viral genome, and cutting the viral genome in order to incapacitate the virus. The presented methods allows for targeted disruption of viral genomic function or, in a preferred embodiment, digestion of viral nucleic acid via multiple breaks caused by targeting multiple sites for endonuclease action in the viral genome. Aspects of the invention provide for transfection of a CRISPR/gRNA/Cas9 complex cocktail to completely suppressed viral proliferation. Additional aspects and advantages of the invention will be apparent upon consideration of the following detailed description thereof.

In certain aspects, the invention provides a method for treating a cell. The method includes the steps of: obtaining a cell from a donor; delivering the RNP to the cell; forming a ribonucleoprotein (RNP) that includes a nuclease and an RNA; and cleaving viral nucleic acid within the cell with the RNP. The method may include providing the cell for transplantation into a patient. Alternatively, the method may be used for research.

The delivering may include electroporation, or the RNP may be packaged in a liposome for the delivering. In some embodiments, the viral nucleic acid will exist as an episomal viral genome, i.e., an episome or episomal vector, of a virus. The RNA has a portion that is substantially complementary to a target within a viral nucleic acid and preferably not substantially complementary to any location on a human genome. In the preferred embodiments, the virus is a herpes family virus such as one selected from the group consisting of HSV-1, HSV-2, Varicella zoster virus, Epstein-Barr virus, and Cytomegalovirus. The virus may be in a latent stage in the cell.

In a preferred embodiment, the nuclease is a Crisper-associated protein such as, preferably, Cas9. The RNA may be a single guide RNA (sgRNA) (providing the functionality of crRNA and tracrRNA). In the preferred embodiment, the nuclease and the RNA are delivered to the cell as the RNP.

In some embodiments, the patient is a pre-determined person who has a human leukocyte antigen (HLA) type matched to the donor. The patient may be the donor. The cell may be a hematopoietic stem cell (e.g., obtained from the donor's bone marrow or peripheral blood). In preferred embodiments, the cell has the viral nucleic acid therein, and the method further comprises cleaving the viral nucleic acid using the nuclease.

The method may include delivering the RNP to a plurality of cells from the donor, culturing the plurality of cells, and selecting the cell from among the plurality of cells based on successful cleavage of the viral nucleic acid. Selecting the cell may include using a fluorescent marker delivered with the nuclease.

Aspects of the invention provide a method for treating a cell to remove foreign nucleic acid. The method comprises: forming a ribonucleoprotein (RNP) that includes a nuclease and an RNA; obtaining a cell from a donor; delivering the RNP to the cell; and cleaving viral nucleic acid within the cell with the RNP. The nuclease may be a CRISPR-associated protein such as is Cas9. The RNA has a portion that is substantially complementary to a target within the viral nucleic acid and not substantially complementary to any location on a human genome. The method may include providing the cell for transplantation into a patient. In some embodiments, the delivering is performed in vitro. Preferably, the foreign nucleic acid comprises viral nucleic acid. In certain embodiments the cell is a hematopoietic stem cell. Optionally the cell is part of a culture of cells and the cells are provided for use in a hematopoietic stem cell transplant (HSCT).

The delivering may include electroporation. The delivering may include packaging the RNP in a liposome.

In preferred embodiments, the virus is a herpes family virus (e.g., HSV-1, HSV-2, Varicella zoster virus, Epstein-Barr virus, or Cytomegalovirus). In some embodiments, the virus is in a latent stage in the cell. The method may include delivering the RNP to a plurality of cells from the donor, culturing the plurality of cells, and selecting the cell from among the plurality of cells based on successful cleavage of the viral nucleic acid.

In certain aspects, the invention provides a composition for treating a cell to remove foreign nucleic acid. The composition comprises: a ribonucleoprotein (RNP) that includes a nuclease and an RNA, wherein the RNA has a portion that is substantially complementary to a target within a non-human nucleic acid and not substantially complementary to any location on a human genome, wherein the RNA guides the nuclease to cleave the non-human nucleic acid. Preferably the nuclease is a CRISPR-associated protein. In preferred embodiments, the non-human nucleic acid comprises a viral nucleic acid from a virus. In certain embodiments, the CRISPR-associated protein is Cas9 and/or the virus is a herpes family virus such as HSV-1, HSV-2, Varicella zoster virus, Epstein-Barr virus, or Cytomegalovirus. The RNP may be enveloped in a liposome.

### BRIEF DESCRIPTION OF THE DRAWINGS

FIG. 1 depicts a flow chart of a method of the invention.

FIG. 2 depicts a scheme of CRISPR/Cas plasmids.

FIG. 3 shows a graph of the effect of oriP on transfection efficiency in Raji cells.

FIG. 4 depicts a CRISPR guide RNA targets along the EBV reference genome.

FIG. 5 depicts genome context around guide RNA sgEBV2 and PCR primer locations.

FIG. 6 depicts a large deletion induced by sgEBV2.

FIG. 7 depicts genome around guide RNA sgEBV3/4/5 and PCR primers.

FIG. 8 depicts large deletions induced by sgEBV3/5 and sgEBV4/5.

FIG. 9 depicts Sanger sequencing confirmed cleavage and repair 8 days after treatment.

FIG. 10 depicts Sanger sequencing confirmed genome cleavage and repair ligation 8 days after sgEBV4/5 treatment.

FIG. 11 depicts cell proliferation curves after different CRISPR treatments.

FIG. 12 depicts flow cytometry scattering signals before sgEBV1-7 treatments.

FIG. 13 depicts flow cytometry scattering signals 5 days after sgEBV1-7 treatments.

FIG. 14 depicts flow cytometry scattering signals 8 days after sgEBV1-7 treatments.

FIG. 15 gives staining results before sgEBV1-7 treatments.

FIG. 16 gives staining results 5 days after sgEBV1-7 treatments.

FIG. 17 gives staining results 8 days after sgEBV1-7 treatments.

FIG. 18 shows microscopy revealed apoptotic morphology after sgEBV1-7 treatment.

FIG. 19 shows microscopy revealed apoptotic morphology after sgEBV1-7 treatment.

FIG. 20 depicts nuclear morphology before sgEBV1-7 treatment.

FIG. 21 depicts nuclear morphology after sgEBV1-7 treatment.

FIG. 22 depicts nuclear morphology after sgEBV1-7 treatment.

FIG. 23 depicts nuclear morphology after sgEBV1-7 treatment.

FIG. 24 depicts EBV load after different CRISPR treatments by digital PCR.

FIG. 25 depicts microscopy of captured single cells for whole-genome amplification.

FIG. 26 depicts microscopy of captured single cells for whole-genome amplification.

FIG. 27 depicts EBV quantitative PCR Cₜ values from single cells before treatment.

FIG. 28 depicts of EBV quantitative PCR Cₜ values from single live cells.

FIG. 29 represents SURVEYOR assay of EBV CRISPR.

FIG. 30 shows CRISPR cytotoxicity test with EBV-negative Burkitt's lymphoma DG-75.

FIG. 31 shows CRISPR cytotoxicity test with primary human lung fibroblast IMR-90.

FIG. 32 shows a method for treating a cell.

FIG. 33 diagrams an experimental design.

FIG. 34 shows EBV+cancer cell survival for 6 days post-treatment.

FIG. 35 shows the percent of each cell population at day 6 post-treatment.

FIG. 36 shows the percent cell survival for 3 days after treatment.

### DETAILED DESCRIPTION

Certain diseases may be curable by a procedure known as hematopoetic stem cell transplantation (HSCT), which replaces a patient's HPCs. Replacement of stem cells has been achieved clinically for decades, as a treatment strategy for a variety of cancers and immunodeficiencies with moderate, but increasing success. HSCT typically includes transplantation of mixed hematopoietic populations that include HSCs and other cells, such as T cells. One limiting factor that is problematic is finding a donor that is HLA type matched to the patient. Since the potential donor pool is small, it would be unfortunate if the rare donor (e.g., a family member) had a viral infection. The invention generally relates to methods for generating viral-free cells for transplantation using a nuclease. Methods of the invention are used to incapacitate or disrupt viruses within a cell by systematically causing large or repeated insertions or deletions in the genome, reducing the probability of reconstructing the full genome. The insertions or deletions in the genome incapacitates or destroys the virus. In some embodiments, the nuclease may be Cas9. In some embodiments, the nuclease is guided by a sequence, such as a guided RNA, that is complementary to. The viral-free cells may then be used for transplantation, for example, in a bone marrow transplant. Thus, methods of the invention may be used to select and isolate viral-free hematopoietic stem cells for transplantation.

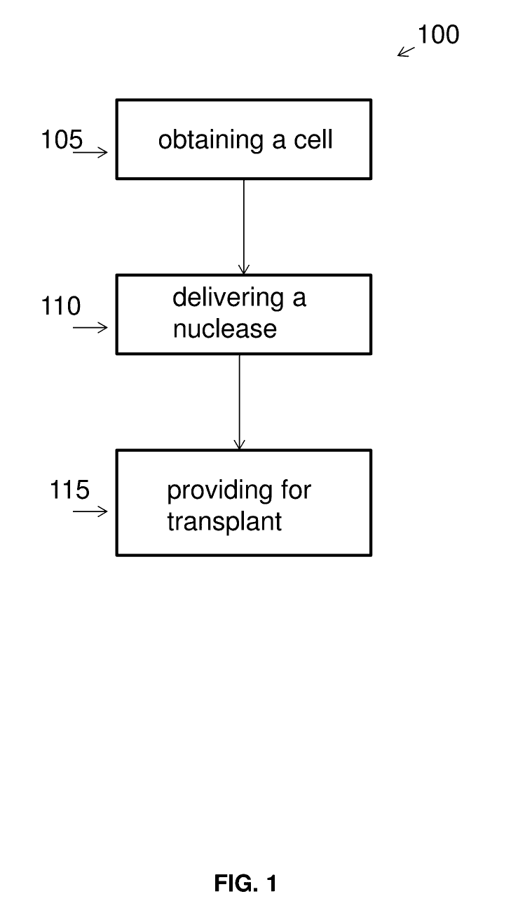

FIG. 1 depicts a flow chart of the method of the invention. In general, the method 100 comprises obtaining a cell 105 from a donor. A nuclease is then delivered to the cell 110, where the nuclease targets one or more portions of a virus genome within the cell. The nuclease is able to bind to and alter the viral genome. The viral-free cell is then provided for transplant 115. The providing for transplant step 115 may be a therapeutic process, such as a bone marrow transplant.

i. Obtaining Cells

Cells for use in the methods of the invention may be obtained from any suitable source. In a preferred embodiment, cells are obtained from a donor, who may be chosen based on being a suitable donor for a patient who will need a bone marrow transplant or other infusion of HSCs. Preferably, the donor is a known family member of the patient, and may even be the patient him- or her-self. For example, a patient may provide their own cells for later delivery in a transplant procedure. E.g., cells may be obtained from an umbilical cord sample taken from the patient and stored, and then treated according to methods of the invention prior to transplant/implantation into the patient.

Any type of cell may be used in the methods of the invention. Cells may be eukaryote, prokaryote, mammalian, human, etc. In some embodiments, stem cells are used in the methods of the invention. Stem cells may be obtained from a stem cell bank, which are ultimately derived from a donor, or directly from a donor. Stem cells may be harvested, purified, and treated by any known method in the art.

Stem cells may be harvested from a donor by any known methods in the art. For example, U.S. Pub. 2013/0149286 details procedures for obtaining and purifying stem cells from mammalian cadavers. Stem cells may be harvested from a human by bone marrow harvest or peripheral blood stem cell harvest, both of which are well known techniques in the art. After stem cells have been obtained from the source, such as from certain tissues of the donor, they may be cultured using stem cell expansion techniques. Stem cell expansion techniques are disclosed in U.S. Pat. No. 6,326,198 to Emerson et al., entitled “Methods and compositions for the ex vivo replication of stem cells, for the optimization of hematopoietic progenitor cell cultures, and for increasing the metabolism, GM-CSF secretion and/or IL-6 secretion of human stromal cells,” issued Dec. 4, 2001; U.S. Pat. No. 6,338,942 to Kraus et al., entitled “Selective expansion of target cell populations,” issued Jan. 15, 2002; and U.S. Pat. No. 6,335,195 to Rodgers et al., entitled “Method for promoting hematopoietic and mesenchymal cell proliferation and differentiation,” issued Jan. 1, 2002, which are hereby incorporated by reference in their entireties. In some embodiments, stem cells obtained from the donor are cultured in order to expand the population of stem cells. In other preferred embodiments, stem cells collected from donor sources are not expanded using such techniques. Standard methods can be used to cyropreserve the stem cells.

In embodiments of the invention, either embryonic or adult stem cells may be used. Adult stem cells, also known as somatic stem cells, may be found in organs and tissues of the donor. For example, the central nervous system, bone marrow, peripheral blood, blood vessels, umbilical cordon blood, skeletal muscle, epidermis of the skin, dental pulp, heart, gut, liver, pancreas, lung, adipose tissue, ovarian epithelium, retina, cornea and testis. Somatic stem cells include, but are not limited to, mesenchymal stem cells, hematopoietic stem cells, skin stem cells, and adipose-derived stromal stem cells. The stem cells may be undifferentiated, or they may be differentiated.

ii. Nuclease

Methods of the invention include using a programmable or targetable nuclease to specifically target viral nucleic acid for destruction. Any suitable targeting nuclease can be used including, for example, zinc-finger nucleases (ZFNs), transcription activator-like effector nucleases (TALENs), clustered regularly interspaced short palindromic repeat (CRISPR) nucleases, meganucleases, other endo- or exo-nucleases, or combinations thereof. See Schiffer, 2012, Targeted DNA mutagenesis for the cure of chronic viral infections, J Virol 88(17):8920-8936, incorporated by reference.

The nuclease targets a portion of a virus's genome to alter or destroy the genome. Once incapacitate or destroyed, the cell is deemed virus free or viral-free. Although fragments or portions of the virus's genome may remain in the cell, the antiviral endonuclease disrupts the viral genome so that the virus is no longer able to replicate, recombine, or infect a host with the virus. In some embodiments, the antiviral endonuclease may be a CRISPR. CRISPR (Clustered Regularly Interspaced Short Palindromic Repeats) is found in bacteria and is believed to protect the bacteria from phage infection. It has recently been used as a means to alter gene expression in eukaryotic DNA, but has not been proposed as an anti-viral therapy or more broadly as a way to disrupt genomic material. Rather, it has been used to introduce insertions or deletions as a way of increasing or decreasing transcription in the DNA of a targeted cell or population of cells. See for example, Horvath et al., Science (2010) 327:167-170; Terns et al., Current Opinion in Microbiology (2011) 14:321-327; Bhaya et al. Annu Rev Genet (2011) 45:273-297; Wiedenheft et al. Nature (2012) 482:331-338); Jinek M et al. Science (2012) 337:816-821; Cong L et al. Science (2013) 339:819-823; Jinek M et al. (2013) eLife 2:e00471; Mali P et al. (2013) Science 339:823-826; Qi L S et al. (2013) Cell 152:1173-1183; Gilbert L A et al. (2013) Cell 154:442-451; Yang H et al. (2013) Cell 154:1370-1379; and Wang H et al. (2013) Cell 153:910-918).

In an aspect of the invention, the Cas9 endonuclease causes a double strand break in at least two locations in the genome. These two double strand breaks cause a fragment of the genome to be deleted. Even if viral repair pathways anneal the two ends, there will still be a deletion in the genome. One or more deletions using the mechanism will incapacitate the viral genome. The result is that the cell will be free of viral infection.

In embodiments of the invention, nucleases cleave the genome of the target virus. A nuclease is an enzyme capable of cleaving the phosphodiester bonds between the nucleotide subunits of nucleic acids. Endonucleases are enzymes that cleave the phosphodiester bond within a polynucleotide chain. Some, such as Deoxyribonuclease I, cut DNA relatively nonspecifically (without regard to sequence), while many, typically called restriction endonucleases or restriction enzymes, cleave only at very specific nucleotide sequences. In a preferred embodiment of the invention, a ribonucleoprotein including a Cas9 nuclease is incorporated into the compositions and methods of the invention, however, it should be appreciated that any nuclease may be utilized.

In preferred embodiments of the invention, the Cas9 nuclease is used to cleave the genome. The Cas9 nuclease is capable of creating a double strand break in the genome. The Cas9 nuclease has two functional domains: RuvC and HNH, each cutting a different strand. When both of these domains are active, the Cas9 causes double strand breaks in the genome.

In some embodiments of the invention, insertions into the genome can be designed to cause incapacitation, or altered genomic expression. Additionally, insertions/deletions are also used to introduce a premature stop codon either by creating one at the double strand break or by shifting the reading frame to create one downstream of the double strand break. Any of these outcomes of the NHEJ repair pathway can be leveraged to disrupt the target gene. The changes introduced by the use of the CRISPR/gRNA/Cas9 system are permanent to the genome.

In some embodiments of the invention, at least one insertion is caused by the CRISPR/gRNA/Cas9 complex. In a preferred embodiment, numerous insertions are caused in the genome, thereby incapacitating the virus. In an aspect of the invention, the number of insertions lowers the probability that the genome may be repaired.

In some embodiments of the invention, at least one deletion is caused by the CRISPR/gRNA/Cas9 complex. In a preferred embodiment, numerous deletions are caused in the genome, thereby incapacitating the virus. In an aspect of the invention, the number of deletions lowers the probability that the genome may be repaired. In a highly-preferred embodiment, the CRISPR/Cas9/gRNA system of the invention causes significant genomic disruption, resulting in effective destruction of the viral genome, while leaving the genome intact.

In some embodiments of the invention, a template sequence is inserted into the genome. In order to introduce nucleotide modifications to genomic DNA, a DNA repair template containing the desired sequence must be present during HDR. The DNA template is normally transfected into the cell along with the gRNA/Cas9. The length and binding position of each homology arm is dependent on the size of the change being introduced. In the presence of a suitable template, HDR can introduce specific nucleotide changes at the Cas9 induced double strand break.

Some embodiments of the invention may utilize modified version of a nuclease. Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. Similar to the inactive dCas9 (RuvC- and HNH-), a Cas9 nickase is still able to bind DNA based on gRNA specificity, though nickases will only cut one of the DNA strands. The majority of CRISPR plasmids are derived from S. pyogenes and the RuvC domain can be inactivated by a D10A mutation and the HNH domain can be inactivated by an H840A mutation.

A single-strand break, or nick, is normally quickly repaired through the HDR pathway, using the intact complementary DNA strand as the template. However, two proximal, opposite strand nicks introduced by a Cas9 nickase are treated as a double strand break, in what is often referred to as a ‘double nick’ or ‘dual nickase’ CRISPR system. A double-nick induced double strain break can be repaired by either NHEJ or HDR depending on the desired effect on the gene target. At these double strain breaks, insertions and deletions are caused by the CRISPR/Cas9 complex. In an aspect of the invention, a deletion is caused by positioning two double strand breaks proximate to one another, thereby causing a fragment of the genome to be deleted.

As versatile as the Cas9 protein is (as either a nuclease, nickase or platform), it may require the targeting specificity of a gRNA in order to act. As discussed below, guide RNAs or single guide RNAs may be specifically designed to target a virus genome.

In some embodiments of the invention, the nuclease is used in conjunction with a guided sequence. In some aspects, the guided sequence is a guided RNA. For example, a CRISPR/Cas9 gene editing complex of the invention works optimally with a guide RNA that targets the viral genome. Guide RNA (gRNA) or single guide RNA (sgRNA) leads the CRISPR/Cas9 complex to the viral genome in order to cause viral genomic disruption. In an aspect of the invention, CRISPR/Cas9/gRNA complexes are designed to target specific viruses within a cell. It should be appreciated that any virus can be targeted using the composition of the invention. Identification of specific regions of the virus genome aids in development and designing of CRISPR/Cas9/gRNA complexes.

In an aspect of the invention, the CRISPR/Cas9/gRNA complexes are designed to target latent viruses within a cell. Once transfected within a cell, the CRISPR/Cas9/gRNA complexes cause repeated insertions or deletions to render the genome incapacitated, or due to number of insertions or deletions, the probability of repair is significantly reduced.

TALENs uses a nonspecific DNA-cleaving nuclease fused to a DNA-binding domain that can be to target essentially any sequence. For TALEN technology, target sites are identified and expression vectors are made. Linearized expression vectors (e.g., by Notl) may be used as template for mRNA synthesis. A commercially available kit may be use such as the mMESSAGE mMACHINE SP6 transcription kit from Life Technologies (Carlsbad, Calif.). See Joung & Sander, 2013, TALENs: a wideliy applicable technology for targeted genome editing, Nat Rev Mol Cell Bio 14:49-55.

TALENs and CRISPR methods provide one-to-one relationship to the target sites, i.e. one unit of the tandem repeat in the TALE domain recognizes one nucleotide in the target site, and the crRNA, gRNA, or sgRNA of CRISPR/Cas system hybridizes to the complementary sequence in the DNA target. Methods can include using a pair of TALENs or a Cas9 protein with one gRNA to generate double-strand breaks in the target. The breaks are then repaired via non-homologous end-joining or homologous recombination (HR).

ZFN may be used to cut viral nucleic acid. Briefly, the ZFN method includes introducing into the infected host cell at least one vector (e.g., RNA molecule) encoding a targeted ZFN and, optionally, at least one accessory polynucleotide. See, e.g., U.S. Pub. 2011/0023144 to Weinstein, incorporated by reference. The cell is incubated to allow expression of the ZFN, wherein a double-stranded break is introduced into the targeted chromosomal sequence by the ZFN. In some embodiments, a donor polynucleotide or exchange polynucleotide is introduced. Swapping a portion of the viral nucleic acid with irrelevant sequence can fully interfere transcription or replication of the viral nucleic acid. Target DNA along with exchange polynucleotide may be repaired by an error-prone non-homologous end-joining DNA repair process or a homology-directed DNA repair process.

Typically, a ZFN comprises a DNA binding domain (i.e., zinc finger) and a cleavage domain (i.e., nuclease) and this gene may be introduced as mRNA (e.g., 5′ capped, polyadenylated, or both). Zinc finger binding domains may be engineered to recognize and bind to any nucleic acid sequence of choice. See, e.g., Qu et al., 2013, Zinc-finger-nucleases mediate specific and efficient excision of HIV-1 proviral DAN from infected and latently infected human T cells, Nucl Ac Res 41(16):7771-7782, incorporated by reference. An engineered zinc finger binding domain may have a novel binding specificity compared to a naturally-occurring zinc finger protein. Engineering methods include, but are not limited to, rational design and various types of selection. A zinc finger binding domain may be designed to recognize a target DNA sequence via zinc finger recognition regions (i.e., zinc fingers). See for example, U.S. Pat. Nos. 6,607,882; 6,534,261 and 6,453,242, incorporated by reference. Exemplary methods of selecting a zinc finger recognition region may include phage display and two-hybrid systems, and are disclosed in U.S. Pat. No. 5,789,538; U.S. Pat. No. 5,925,523; U.S. Pat. No. 6,007,988; U.S. Pat. No. 6,013,453; U.S. Pat. No. 6,410,248; U.S. Pat. No. 6,140,466; U.S. Pat. No. 6,200,759; and U.S. Pat. No. 6,242,568, each of which is incorporated by reference.

A ZFN also includes a cleavage domain. The cleavage domain portion of the ZFNs may be obtained from any suitable endonuclease or exonuclease such as restriction endonucleases and homing endonucleases. See, for example, Belfort & Roberts, 1997, Homing endonucleases: keeping the house in order, Nucleic Acids Res 25(17):3379-3388. A cleavage domain may be derived from an enzyme that requires dimerization for cleavage activity. Two ZFNs may be required for cleavage, as each nuclease comprises a monomer of the active enzyme dimer. Alternatively, a single ZFN may comprise both monomers to create an active enzyme dimer. Restriction endonucleases present may be capable of sequence-specific binding and cleavage of DNA at or near the site of binding. Certain restriction enzymes (e.g., Type IIS) cleave DNA at sites removed from the recognition site and have separable binding and cleavage domains. For example, the Type IIS enzyme FokI, active as a dimer, catalyzes double-stranded cleavage of DNA, at 9 nucleotides from its recognition site on one strand and 13 nucleotides from its recognition site on the other. The FokI enzyme used in a ZFN may be considered a cleavage monomer. Thus, for targeted double-stranded cleavage using a FokI cleavage domain, two ZFNs, each comprising a FokI cleavage monomer, may be used to reconstitute an active enzyme dimer. See Wah, et al., 1998, Structure of FokI has implications for DNA cleavage, PNAS 95:10564-10569; U.S. Pat. No. 5,356,802; U.S. Pat. No. 5,436,150; U.S. Pat. No. 5,487,994; U.S. Pub. 2005/0064474; U.S. Pub. 2006/0188987; and U.S. Pub. 2008/0131962, each incorporated by reference.

Virus targeting using ZFN may include introducing at least one donor polynucleotide comprising a sequence into the cell. A donor polynucleotide preferably includes the sequence to be introduced flanked by an upstream and downstream sequence that share sequence similarity with either side of the site of integration in the chromosome. The upstream and downstream sequences in the donor polynucleotide are selected to promote recombination between the chromosomal sequence of interest and the donor polynucleotide. Typically, the donor polynucleotide will be DNA. The donor polynucleotide may be a DNA plasmid, a bacterial artificial chromosome (BAC), a yeast artificial chromosome (YAC), a viral vector, a linear piece of DNA, a PCR fragment, a naked nucleic acid, and may employ a delivery vehicle such as a liposome. The sequence of the donor polynucleotide may include exons, introns, regulatory sequences, or combinations thereof. The double stranded break is repaired via homologous recombination with the donor polynucleotide such that the desired sequence is integrated into the chromosome. In the ZFN-mediated process for modifying a chromosomal sequence, a double stranded break introduced into the chromosomal sequence by the ZFN is repaired, via homologous recombination with the exchange polynucleotide, such that the sequence in the exchange polynucleotide may be exchanged with a portion of the chromosomal sequence. The presence of the double stranded break facilitates homologous recombination and repair of the break. The exchange polynucleotide may be physically integrated or, alternatively, the exchange polynucleotide may be used as a template for repair of the break, resulting in the exchange of the sequence information in the exchange polynucleotide with the sequence information in that portion of the chromosomal sequence. Thus, a portion of the viral nucleic acid may be converted to the sequence of the exchange polynucleotide. ZFN methods can include using a vector to deliver a nucleic acid molecule encoding a ZFN and, optionally, at least one exchange polynucleotide or at least one donor polynucleotide to the infected cell.

Meganucleases are endodeoxyribonucleases characterized by a large recognition site (double-stranded DNA sequences of 12 to 40 base pairs); as a result this site generally occurs only once in any given genome. For example, the 18-base pair sequence recognized by the I-SceI meganuclease would on average require a genome twenty times the size of the human genome to be found once by chance (although sequences with a single mismatch occur about three times per human-sized genome). Meganucleases are therefore considered to be the most specific naturally occurring restriction enzymes. Meganucleases can be divided into five families based on sequence and structure motifs: LAGLIDADG, GIY-YIG, HNH, His-Cys box and PD-(D/E)XK. The most well studied family is that of the LAGLIDADG proteins, which have been found in all kingdoms of life, generally encoded within introns or inteins although freestanding members also exist. The sequence motif, LAGLIDADG, represents an essential element for enzymatic activity. Some proteins contained only one such motif, while others contained two; in both cases the motifs were followed by ˜75-200 amino acid residues having little to no sequence similarity with other family members. Crystal structures illustrates mode of sequence specificity and cleavage mechanism for the LAGLIDADG family: (i) specificity contacts arise from the burial of extended β-strands into the major groove of the DNA, with the DNA binding saddle having a pitch and contour mimicking the helical twist of the DNA; (ii) full hydrogen bonding potential between the protein and DNA is never fully realized; (iii) cleavage to generate the characteristic 4-nt 3′-OH overhangs occurs across the minor groove, wherein the scissile phosphate bonds are brought closer to the protein catalytic core by a distortion of the DNA in the central “4-base” region; (iv) cleavage occurs via a proposed two-metal mechanism, sometimes involving a unique “metal sharing” paradigm; (v) and finally, additional affinity and/or specificity contacts can arise from “adapted” scaffolds, in regions outside the core α/β fold. See Silva et al., 2011, Meganucleases and other tools for targeted genome engineering, Curr Gene Ther 11(1):11-27, incorporated by reference.

iii. Apoptotic Pathway

In cases where a small number of cells are infected and it would suffice to ablate the entire cell (as well as the latent viral genome), an aspect of the invention contemplates administration of a vector containing a promoter which is active in the latent viral state, wherein the promoter drives a cell-killing gene. HSV is a particularly interesting target for this approach as it has been estimated that only thousands to tens of thousands neurons are latently infected. See Hoshino et al., 2008, The number of herpes simplex virus-infected neurons and the number of viral genome copies per neuron correlate with latent viral load in ganglia, Virology 372(1):56-63, incorporated by reference. Examples of cell-killing genes include both (1) targetable nucleases that are targeted to the cell genome; and (2) apoptosis effectors such as BAX and BAK and proteins that destroy the integrity of the cell or mitochondrial membrane, such as alpha hemolysin. (Bayles, “Bacterial programmed cell death: making sense of a paradox,” Nature Reviews Microbiology 12 pp. 63-69 (2014)). Having a promoter that is only activated in latently infected cells could be used not only in this context but also be used to increase selectivity of nuclease therapy by making activity specific to infected cells; an example of such a promoter is Latency-Associated Promoter 1, or “LAP1”. (Preston and Efstathiou, “Molecular Basis of HSV Latency and Reactivation”, in Human Herpesviruses: Biology, Therapy and Immunoprophylaxis 2007.) In some embodiments, the invention provides methods and therapeutics that can be used to cause the death of host cells but only those cells that are infected. For example, the treatment can include delivering a gene for a protein that causes cell death, where the gene is under control of a viral regulatory element such as a promoter from the genome of the infecting virus or the gene is encoded in a vector that includes a viral origin of replication. Where the virus is present, the gene will be expressed and the gene product will cause the death of the cell. The gene can code for a protein important in apoptosis, or the gene can code for a nuclease that digests the host genome.

The apoptotic embodiments may be used to remove infected cells from within a sample that contains a mix of infected and uninfected cells. Using a targetable nuclease, a composition may be provided that includes a viral-driven promoter, a targetable nuclease, and guide RNAs that target the cellular (e.g., human) genome. In the presence of the virus, the nuclease will kill the cells. The sample will be left containing only uninfected cells.

An apoptosis protein may be used as the therapeutic. The therapeutic may be provided encoded within a vector, in which the vector also encodes a sequence that causes the therapeutic to be expressed within a cell that is infected by a virus. The sequence may be a regulatory element (e.g., a promoter and an origin of replication) from the genome of the virus. The therapeutic may provide a mechanism that selectively causes death of virus-infected cells. For example, a protein may be used that restores a deficient apoptotic pathway in the cell. The gene may be, for example, BAX, BAK, BCL-2, or alpha-hemolysin. Preferably, the therapeutic induces apoptosis in the cell that is infected by the virus and does not induce apoptosis in an uninfected cell.

In some embodiments, the invention provides a composition that includes a viral vector, plasmid, or other coding nucleic acid that encodes at least one gene that promotes apoptosis and at least one promoter associated a viral genome. Apoptosis regulator Bcl-2 is a family of proteins that govern mitochondrial outer membrane permeabilization (MOMP) and include pro-apoptotic proteins such as Bax, BAD, Bak, Bok, Bcl-rambo, Bcl-xs and BOK/Mtd.

Apoptosis regulator BAX, also known as bcl-2-like protein 4, is a protein that in humans is encoded by the BAX gene. BAX is a member of the Bcl-2 gene family. This protein forms a heterodimer with BCL2, and functions as an apoptotic activator. This protein is reported to interact with, and increase the opening of, the mitochondrial voltage-dependent anion channel (VDAC), which leads to the loss in membrane potential and the release of cytochrome c.

Bcl-2 homologous antagonist/killer is a protein that in humans is encoded by the BAK1 gene on chromosome 6. This protein localizes to mitochondria, and functions to induce apoptosis. It interacts with and accelerates the opening of the mitochondrial voltage-dependent anion channel, which leads to a loss in membrane potential and the release of cytochrome c.

Human genes encoding proteins that belong to this family include: BAK1, BAX, BCL2, BCL2A1, BCL2L1, BCL2L2, BCL2L10, BCL2L13, BCL2L14, BOK, and MCL1.

iv. Target Specificity

A nuclease may use the targeting specificity of a gRNA in order to cleave viral nucleic acid without interfering with the genome or function of the HSCs or cells to be transplanted. The nuclease may cleave a single strand of nucleic acid or cause a double strain break in the nucleic acid.

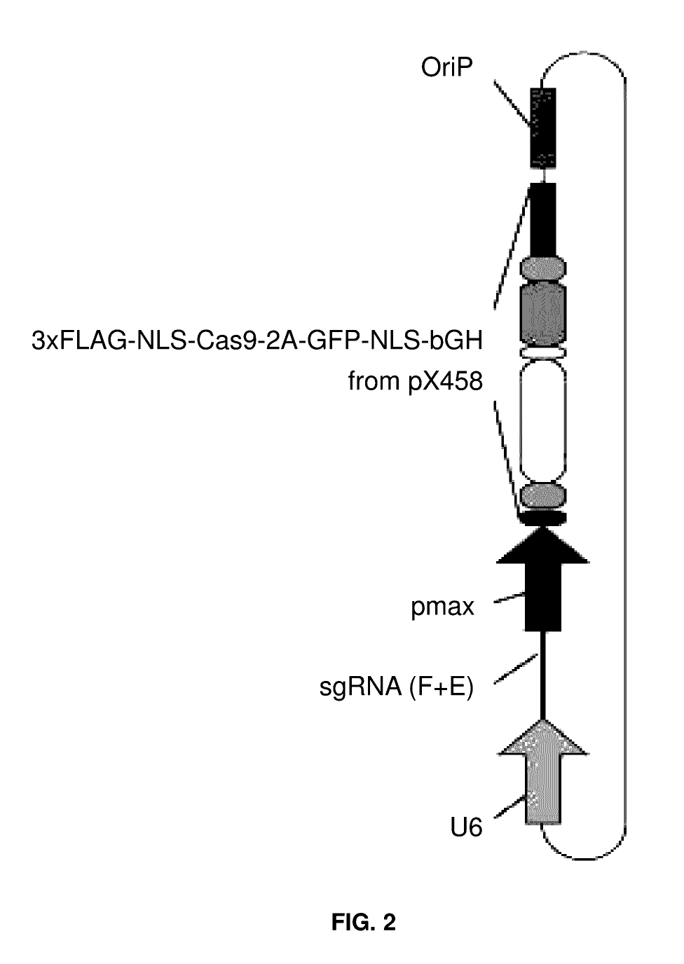

As an example, the Epstein-Barr virus (EBV), also called human herpesvirus 4 (HHV-4) is inactivated in cells by a CRISPR/Cas9/gRNA complex of the invention. EBV is a virus of the herpes family, and is one of the most common viruses in humans. The virus is approximately 122 nm to 180 nm in diameter and is composed of a double helix of DNA wrapped in a protein capsid. In this example, the Raji cell line serves as an appropriate in vitro model. The Raji cell line is the first continuous human cell line from hematopoietic origin and cell lines produce an unusual strain of Epstein-Barr virus while being one of the most extensively studied EBV models. To target the EBV genomes in the Raji cells, a CRISPR/Cas9 complex with specificity for EBV is needed. The design of EBV-targeting CRISPR/Cas9 plasmids consisting of a U6 promoter driven chimeric guide RNA (sgRNA) and a ubiquitous promoter driven Cas9 that were obtained from Addgene, Inc. Commercially available guide RNAs and Cas9 nucleases may be used with the present invention. An EGFP marker fused after the Cas9 protein allowed selection of Cas9-positive cells (FIG. 2).

In an aspect of the invention, guide RNAs are designed, whether or not commercially purchased, to target a specific viral genome. The viral genome is identified and guide RNA to target selected portions of the viral genome are developed and incorporated into the composition of the invention. In an aspect of the invention, a reference genome of a particular strain of the virus is selected for guide RNA design.

For example, guide RNAs that target the EBV genome are a component of the system in the present example. In relation to EBV, for example, the reference genome from strain B95-8 was used as a design guide. Within a genome of interest, such as EBV, selected regions, or genes are targeted. For example, six regions can be targeted with seven guide RNA designs for different genome editing purposes (FIG. 4 and Table 1). Additional information such as primer design is shown in Wang and Quake, 2014, RNA-guided endonuclease provides a therapeutic strategy to cure latent herpesviridae infection, PNAS 111(36):13157-13162 and in the Supporting Information to that article published online at the PNAS website, and the contents of both of those documents are incorporated by reference for all purposes.

TABLE 1
Guide RNA target sequences

| | | |
|---|---|---|
| sgEBV1 | GCCCTGGACCAACCCGGCCC | (SEQ ID NO: 1) |
| sgEBV2 | GGCCGCTGCCCCGCTCCGGG | (SEQ ID NO: 2) |
| sgEBV3 | GGAAGACAATGTGCCGCCA | (SEQ ID NO: 3) |
| sgEBV4 | TCTGGACCAGAAGGCTCCGG | (SEQ ID NO: 4) |
| sgEBV5 | GCTGCCGCGGAGGGTGATGA | (SEQ ID NO: 5) |
| sgEBV6 | GGTGGCCCACCGGGTCCGCT | (SEQ ID NO: 6) |
| sgEBV7 | GTCCTCGAGGGGGCCGTCGC | (SEQ ID NO: 7) |

In relation to EBV, EBNA1 is the only nuclear Epstein-Barr virus (EBV) protein expressed in both latent and lytic modes of infection. While EBNA1 is known to play several important roles in latent infection, EBNA1 is crucial for many EBV functions including gene regulation and latent genome replication. Therefore, guide RNAs sgEBV4 and sgEBV5 were selected to target both ends of the EBNA1 coding region in order to excise this whole region of the genome. These “structural” targets enable systematic digestion of the EBV genome into smaller pieces. EBNA3C and LMP1 are essential for cell transformation, and guide RNAs sgEBV3 and sgEBV7 were designed to target the 5′ exons of these two proteins respectively.

In some embodiments, antiviral endonucleases are introduced into a cell. In some embodiments, CRISPR/Cas9/gRNA complexes are introduced into a cell. A guide RNA is designed to target at least one category of sequences of the viral genome.

In some embodiments, a cocktail of guide RNAs may be introduced into a cell. The guide RNAs are designed to target numerous categories of sequences of the viral genome. By targeting several areas along the genome, the double strand break at multiple locations fragments the genome, lowering the possibility of repair. Even with repair mechanisms, the large deletions render the virus incapacitated.

In some embodiments, several guide RNAs are added to create a cocktail to target different categories of sequences. For example, two, five, seven or eleven guide RNAs may be present in a CRISPR cocktail targeting three different categories of sequences. However, any number of gRNAs may be introduced into a cocktail to target categories of sequences. In preferred embodiments, the categories of sequences are important for genome structure, and infection latency, respectively.

In some aspects of the invention, in vitro experiments allow for the determination of the most essential targets within a viral genome. For example, to understand the most essential targets for effective incapacitation of a genome, subsets of guide RNAs are transfected into model cells. Assays can determine which guide RNAs or which cocktail is the most effective at targeting essential categories of sequences.

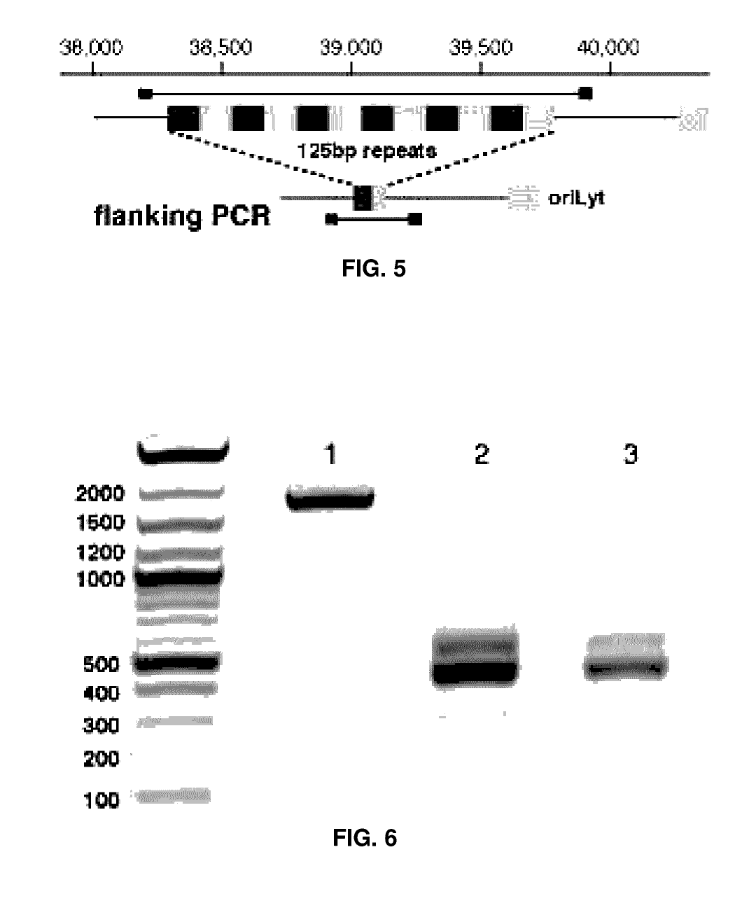

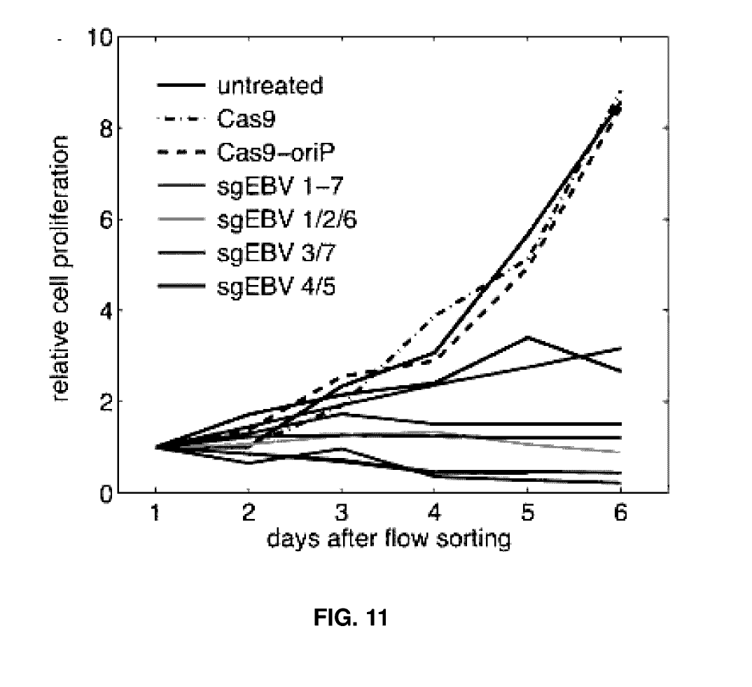

For example, in the case of the EBV genome targeting, seven guide RNAs in the CRISPR cocktail targeted three different categories of sequences which are identified as being important for EBV genome structure, cell transformation, and infection latency, respectively. To understand the most essential targets for effective EBV treatment, Raji cells were transfected with subsets of guide RNAs. Although sgEBV4/5 reduced the EBV genome by 85%, they could not suppress cell proliferation as effectively as the full cocktail (FIGS. 11-23). Guide RNAs targeting the structural sequences (sgEBV1/2/6) could stop cell proliferation completely, despite not eliminating the full EBV load (26% decrease). Given the high efficiency of genome editing and the proliferation arrest (FIGS. 5-10), it was suspect that the residual EBV genome signature in sgEBV1/2/6 was not due to intact genomes but to free-floating DNA that has been digested out of the EBV genome, i.e. as a false positive.

Once CRISPR/Cas9/gRNA complexes are constructed, the complexes are introduced into a cell. In an aspect of the invention, CRISPR/Cas9/gRNA complexes are designed to not leave intact genomes of a virus after transfection and complexes are designed for efficient transfection.

v. Delivery Vectors

Aspects of the invention allow for CRISPR/Cas9/gRNA to be transfected into cells by various methods, including viral vectors and non-viral vectors. Viral vectors may include retroviruses, lentiviruses, adenoviruses, and adeno-associated viruses. It should be appreciated that any viral vector may be incorporated into the present invention to effectuate delivery of the CRISPR/Cas9/gRNA complex into a cell. Some viral vectors may be more effective than others, depending on the CRISPR/Cas9/gRNA complex designed for digestion or incapacitation. In an aspect of the invention, the vectors contain essential components such as origin of replication, which is necessary for the replication and maintenance of the vector in the cell.

In an aspect of the invention, viral vectors are used as delivery vectors to deliver the complexes into a cell. Use of viral vectors as delivery vectors are known in the art. See for example U.S. Pub. 2009/0017543, the contents of which are incorporated by reference.

A retrovirus is a single-stranded RNA virus that stores its nucleic acid in the form of an mRNA genome (including the 5′ cap and 3′ PolyA tail) and targets a cell as an obligate parasite. In some methods in the art, retroviruses have been used to introduce nucleic acids into a cell. Once inside the cell cytoplasm the virus uses its own reverse transcriptase enzyme to produce DNA from its RNA genome, the reverse of the usual pattern, thus retro (backwards). This new DNA is then incorporated into the cell genome by an integrase enzyme, at which point the retroviral DNA is referred to as a provirus. For example, the recombinant retroviruses such as the Moloney murine leukemia virus have the ability to integrate into the genome in a stable fashion. They contain a reverse transcriptase that allows integration into the genome. Retroviral vectors can either be replication-competent or replication-defective. In some embodiments of the invention, retroviruses are incorporated to effectuate transfection into a cell, however the CRISPR/Cas9/gRNA complexes are designed to target the viral genome.

In some embodiments of the invention, lentiviruses, which are a subclass of retroviruses, are used as viral vectors. Lentiviruses can be adapted as delivery vehicles (vectors) given their ability to integrate into the genome of non-dividing cells, which is the unique feature of lentiviruses as other retroviruses can infect only dividing cells. The viral genome in the form of RNA is reverse-transcribed when the virus enters the cell to produce DNA, which is then inserted into the genome at a random position by the viral integrase enzyme. The vector, now called a provirus, remains in the genome and is passed on to the progeny of the cell when it divides.

As opposed to lentiviruses, adenoviral DNA does not integrate into the genome and is not replicated during cell division. Adenovirus and the related AAV would be potential approaches as delivery vectors since they do not integrate into the cell's genome. In some aspects of the invention, only the viral genome to be targeted is effected by the CRISPR/Cas9/gRNA complexes, and not other genetic material in the cell. Adeno-associated virus (AAV) is a small virus that infects humans and some other primate species. AAV can infect both dividing and non-dividing cells and may incorporate its genome into that of the host cell. For example, because of its potential use as a gene therapy vector, researchers have created an altered AAV called self-complementary adeno-associated virus (scAAV). Whereas AAV packages a single strand of DNA and requires the process of second-strand synthesis, scAAV packages both strands which anneal together to form double stranded DNA. By skipping second strand synthesis scAAV allows for rapid expression in the cell. Otherwise, scAAV carries many characteristics of its AAV counterpart. Methods of the invention may incorporate herpesvirus, poxvirus, alphavirus, or vaccinia virus as a means of delivery vectors.

In certain embodiments of the invention, non-viral vectors may be used to effectuate transfection. Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam and Lipofectin). Cationic and neutral lipids that are suitable for efficient receptor-recognition lipofection of polynucleotides include those of Felgner, U.S. Pat. No. 4,897,355; U.S. Pat. No. 4,946,787; U.S. Pat. No. 5,049,386; and U.S. Pat. No. 5,208,036.

Synthetic vectors are typically based on cationic lipids or polymers which can complex with negatively charged nucleic acids to form particles with a diameter in the order of 100 nm. The complex protects nucleic acid from degradation by nuclease. Moreover, cellular and local delivery strategies have to deal with the need for internalization, release, and distribution in the proper subcellular compartment. In some embodiments of the invention, non-viral vectors are modified to effectuate targeted delivery and transfection. PEGylation (i.e. modifying the surface with polyethyleneglycol) is the predominant method used to reduce the opsonization and aggregation of non-viral vectors.

However, PEG on the surface can decrease the uptake by target cells and reduce the biological activity. Therefore, to attach targeting ligand to the distal end of the PEGylated component is necessary; the ligand is projected beyond the PEG “shield” to allow binding to receptors on the target cell surface. When cationic liposome is used as gene carrier, the application of neutral helper lipid is helpful for the release of nucleic acid, besides promoting hexagonal phase formation to enable endosomal escape. Designing and synthesizing novel cationic lipids and polymers, and covalently or noncovalently binding gene with peptides, targeting ligands, polymers, or environmentally sensitive moieties also attract many attentions for resolving the problems encountered by non-viral vectors. The application of inorganic nanoparticles (for example, metallic nanoparticles, iron oxide, calcium phosphate, magnesium phosphate, manganese phosphate, double hydroxides, carbon nanotubes, and quantum dots) in delivery vectors can be prepared and surface-functionalized in many different ways.

In some embodiments of the invention, targeted controlled-release systems responding to the unique environments of tissues and external stimuli are utilized. Gold nanorods have strong absorption bands in the near-infrared region, and the absorbed light energy is then converted into heat by gold nanorods, the so-called ‘photothermal effect’. Because the near-infrared light can penetrate deeply into tissues, the surface of gold nanorod could be modified with nucleic acids for controlled release. When the modified gold nanorods are irradiated by near-infrared light, nucleic acids are released due to thermo-denaturation induced by the photothermal effect. The amount of nucleic acids released is dependent upon the power and exposure time of light irradiation.

In some embodiments of the invention, liposomes are used to effectuate transfection into a cell or tissue. The pharmacology of a liposomal formulation of nucleic acid is largely determined by the extent to which the nucleic acid is encapsulated inside the liposome bilayer. Encapsulated nucleic acid is protected from nuclease degradation, while those merely associated with the surface of the liposome is not protected. Encapsulated nucleic acid shares the extended circulation lifetime and biodistribution of the intact liposome, while those that are surface associated adopt the pharmacology of naked nucleic acid once they disassociate from the liposome.

In some embodiments, the complexes of the invention are encapsulated in a liposome. Unlike small molecule drugs, nucleic acids cannot cross intact lipid bilayers, predominantly due to the large size and hydrophilic nature of the nucleic acid. Therefore, nucleic acids may be entrapped within liposomes with conventional passive loading technologies, such as ethanol drop method (as in SALP), reverse-phase evaporation method, and ethanol dilution method (as in SNALP).

In some embodiments, linear polyethylenimine (L-PEI) is used as a non-viral vector due to its versatility and comparatively high transfection efficiency. L-PEI is able to efficiently condense, stabilize and deliver nucleic acids in vitro.

Besides ultrasound-mediated delivery, magnetic targeting delivery could be used for delivery. Magnetic nanoparticles are usually entrapped in gene vectors for imaging the delivery of nucleic acid. Nucleic acid carriers can be responsive to both ultrasound and magnetic fields, i.e., magnetic and acoustically active lipospheres (MAALs). The basic premise is that therapeutic agents are attached to, or encapsulated within, a magnetic micro- or nanoparticle. These particles may have magnetic cores with a polymer or metal coating which can be functionalized, or may consist of porous polymers that contain magnetic nanoparticles precipitated within the pores. By functionalizing the polymer or metal coating it is possible to attach, for example, cytotoxic drugs for targeted chemotherapy or therapeutic DNA to correct a genetic defect. Magnetic fields, generally from high-field, high-gradient, rare earth magnets are focused over the target site and the forces on the particles as they enter the field allow them to be captured and extravasated at the target.

Synthetic cationic polymer-based nanoparticles (˜100 nm diameter) have been developed that offer enhanced transfection efficiency combined with reduced cytotoxicity, as compared to traditional liposomes. The incorporation of distinct layers composed of lipid molecules with varying physical and chemical characteristics into the polymer nanoparticle formulation resulted in improved efficiency through better fusion with cell membrane and entry into the cell, enhanced release of molecules inside the cell, and reduced intracellular degradation of nanoparticle complexes.

In some embodiments, the complexes are conjugated to nano-systems, such as liposomes, albumin-based particles, PEGylated proteins, biodegradable polymer-drug composites, polymeric micelles, dendrimers, among others. Davis M E, Chen Z G, Shin D M. Nat Rev Drug Discov. 2008; 7:771-782. In certain embodiments, the complexes of the invention are conjugated to or encapsulated into a liposome or polymerosome for delivery to a cell. For example, liposomal anthracyclines have achieved highly efficient encapsulation, and include versions with greatly prolonged circulation such as liposomal daunorubicin and pegylated liposomal doxorubicin. See Krishna et al., Carboxymethylcellulose-sodium based transdermal drug delivery system for propranolol, J Pharm Pharmacol. 1996 April; 48(4):367-70.

Liposomes and polymerosomes can contain a plurality of solutions and compounds. In certain embodiments, the complexes of the invention are coupled to or encapsulated in polymersomes. As a class of artificial vesicles, polymersomes are tiny hollow spheres that enclose a solution, made using amphiphilic synthetic block copolymers to form the vesicle membrane. Common polymersomes contain an aqueous solution in their core and are useful for encapsulating and protecting sensitive molecules, such as drugs, enzymes, other proteins and peptides, and DNA and RNA fragments. The polymersome membrane provides a physical barrier that isolates the encapsulated material from external materials, such as those found in biological systems. Polymerosomes can be generated from double emulsions by known techniques, see Lorenceau et al., 2005, Generation of Polymerosomes from Double-Emulsions, Langmuir 21(20):9183-6, incorporated by reference.

Some embodiments of the invention provide for a gene gun or a biolistic particle delivery system. A gene gun is a device for injecting cells with genetic information, where the payload may be an elemental particle of a heavy metal coated with plasmid DNA. This technique may also be referred to as bioballistics or biolistics. Gene guns have also been used to deliver DNA vaccines. The gene gun is able to transfect cells with a wide variety of organic and non-organic species, such as DNA plasmids, fluorescent proteins, dyes, etc.

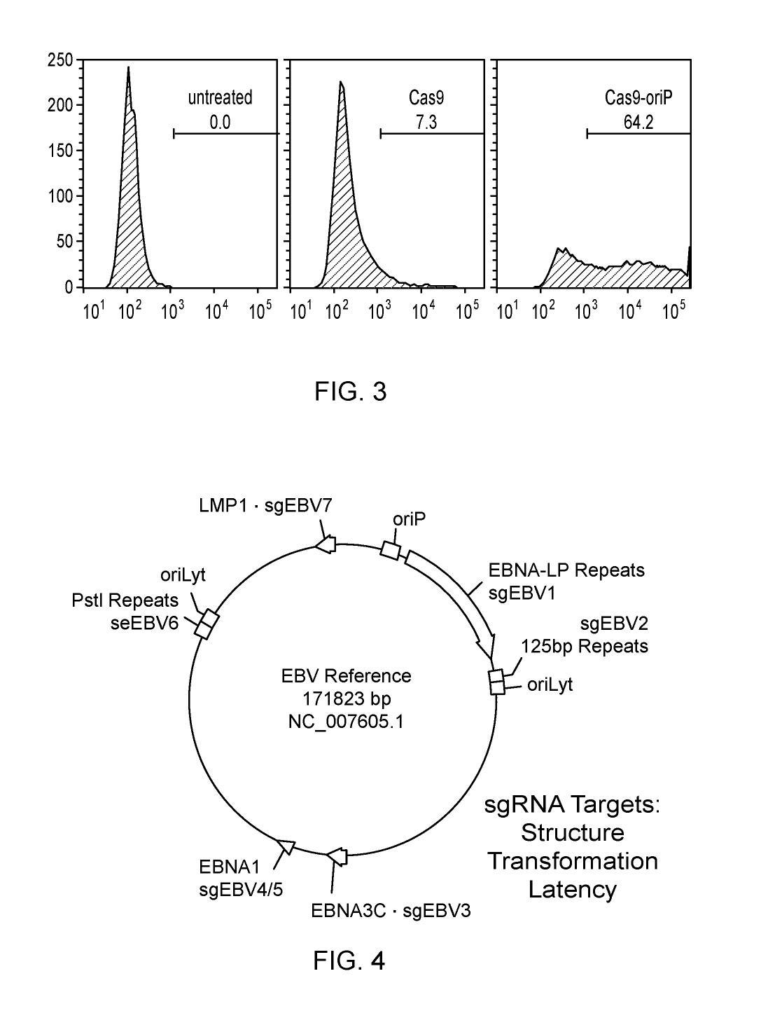

Aspects of the invention provide for numerous uses of delivery vectors. Selection of the delivery vector is based upon the cell or tissue targeted and the specific makeup of the antiviral endonuclease. For example, in the EBV example discussed above, since lymphocytes are known for being resistant to lipofection, nucleofection (a combination of electrical parameters generated by a device called Nucleofector, with cell-type specific reagents to transfer a substrate directly into the cell nucleus and the cytoplasm) was necessitated for DNA delivery into the Raji cells. The Lonza pmax promoter drives Cas9 expression as it offered strong expression within Raji cells. 24 hours after nucleofection, obvious EGFP signals were observed from a small proportion of cells through fluorescent microscopy. The EGFP-positive cell population decreased dramatically, however, <10% transfection efficiency 48 hours after nucleofection was measured (FIG. 3). A CRISPR plasmid that included the EBV origin of replication sequence, oriP yielded a transfection efficiency >60% (FIG. 3).

vi. Cleaving Viral Nucleic Acid

Methods of the invention may be used prophylactically, i.e., to treat all cells prior to transplant, even without knowledge of infection. In some embodiments, a state of infection is known and only infected cells are treated. When an infected cell is treated, the nuclease cleaves the viral nucleic acid.

The viral nucleic acid that is cleaved may be free particles of viral DNA or RNA or may include viral nucleic acid that has been integrated into the host genome. The targeted virus may be in an active or latent stage of infection.

Once inside the cell, the nuclease targets the viral genome. For example, the CRISPR/Cas9/gRNA complex may target the viral genome. In addition to latent infections this invention can also be used to control actively replicating viruses by targeting the viral genome before it is packaged or after it is ejected. In preferred embodiments, the CRISPR/Cas9/gRNA complexes target latent viral genomes, thereby reducing the chances of proliferation. The guided RNA complexes target a determined number of categories of sequences of the viral genome to incapacitate the viral genome. As discussed above, the Cas9 endonuclease causes a double strand break in the viral genome. By targeted several locations along the viral genome and causing not a single strand break, but a double strand break, the genome is effectively cut a several locations along the genome. In a preferred embodiment, the double strand breaks are designed so that small deletions are caused, or small fragments are removed from the genome so that even if natural repair mechanisms join the genome together, the genome is render incapacitated.

In a preferred embodiment of the invention, CRISPR/Cas9/gRNA complexes are transfected into cells containing viral genomes. The gRNAs are designed to localize the Cas9 endonuclease at several locations along the viral genome. The Cas9 endonuclease caused double strand breaks in the genome, causing small fragments to be deleted from the viral genome. Even with repair mechanisms, the deletions render the viral genome incapacitated.

Cells treated with an antiviral endonuclease according to the methods of the invention are then provided for transplantation. A stem cell transplant (sometimes called a bone marrow transplant) is a medical procedure in which diseased bone marrow is replaced by highly specialized stem cells that develop into healthy bone marrow. Methods and procedures for bone marrow transplant are well known in the art. See for example U.S. Pat. No. 6,383,481, entitled, “Method for transplantation of hemopoietic stem cells.” After treatment with antiviral endonuclease, the cell may be stored until used in transplantation.

In some methods of the invention, cells treated with antiviral endonucleases are grown in culture. In some embodiments, laboratory techniques are used to create a population of cells derived from a cell treated or exposed to an antiviral endonuclease. Cell culturing techniques are well known in the art. For example, see U.S. Pub. 2011/0177594 entitled “Stem Cells Culture Systems”; U.S. Pub. 2012/0122213 entitled “Method for Culturing Stem Cells”; and U.S. Pub. 2009/0325294 entitled “Single Pluripotent Stem Cell Culture”. The cells grown from the treated cell may then be stored until use in transplantation. Importantly, the cells grown from the treated cell is free of virus targeted by the antiviral endonuclease.

vii. Delivery to Recipient

Methods of the invention include providing the cell for transplant into the patient. In some embodiments, the treated cells are labeled, stored, shipped, or otherwise readied for medical use. In certain embodiments, methods of the invention include delivering the cell or cells into the body of the patient.

In some embodiments, hematopoietic stem cell transplantation (HSCT) involves the intravenous (IV) infusion of autologous or allogeneic stem cells to reestablish hematopoietic function in patients whose bone marrow or immune system is damaged or defective. Hematopoietic stem cell transplantation (HSCT) requires the extraction (apheresis) of haematopoietic stem cells (HSC) from the patient and storage of the harvested cells in a freezer. The patient is then treated with high-dose chemotherapy with or without radiotherapy with the intention of eradicating the patient's malignant cell population at the cost of partial or complete bone marrow ablation (destruction of patient's bone marrow function to grow new blood cells). The patient's own stored stem cells are then treated with nucleases according to methods of the invention, and then transfused into his/her bloodstream, where they replace destroyed tissue and resume the patient's normal blood cell production.

In some embodiments, allogeneic HSCT, which involves a healthy donor and the patient recipient, incorporate methods of the invention. Allogeneic HSC donors must have a tissue (HLA) type that matches the recipient. Matching is performed on the basis of variability at three or more loci of the HLA gene, and a perfect match at these loci is preferred. Allogeneic transplant donors may be related (usually a closely HLA matched sibling), syngeneic (a monozygotic or ‘identical’ twin of the patient—necessarily extremely rare since few patients have an identical twin, but offering a source of perfectly HLA matched stem cells) or unrelated (donor who is not related and found to have very close degree of HLA matching). Unrelated donors may be found through a registry of bone marrow donors such as the National Marrow Donor Program. In general, by transfusing healthy stem cells to the recipient's bloodstream to reform a healthy immune system, allogeneic HSCTs may improve chances for cure or long-term remission once the immediate transplant-related complications are resolved.

Cells harvested or obtained may be frozen (cryopreserved) for prolonged periods without damaging the cells. In some embodiments, the cells may be harvested from the recipient or donor months or years in advance of the transplant treatment. To cryopreserve HSC, a preservative, DMSO, may be added, and the cells may be cooled very slowly in a controlled-rate freezer to prevent osmotic cellular injury during ice crystal formation. HSC may be stored for years in a cryofreezer, which typically uses liquid nitrogen.

Providing for medical use can include labeling, storing, shipping, or otherwise readying for use. In a preferred embodiment, providing the cells for transplant into the patient includes putting the cells in a container, such as the blood collection tube sold under the trademark VACUTAINER by BD (Franklin Lakes, N.J.) that is labeled with information that can be used to identify the recipient. The container may be stored for a period of time until the cells are needed for transplantation. In some embodiments, providing the cells for transplant into the patient includes holding the cells in a container after delivering a nuclease.

Delivering into the patient may include delivering viral-free cells into a patient by intravenous (IV) infusion. In other embodiments, the viral-free cells may be transplanted into a patient via a surgery, or by placing the sample into a location in the patient's body. In other embodiments, the cells are placed into a patient during a surgical procedure.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

### EXAMPLES

### Example 1

Burkitt's lymphoma cell lines Raji, Namalwa, and DG-75 were obtained from ATCC and cultured in RPMI 1640 supplemented with 10% FBS and PSA, following ATCC recommendation. Human primary lung fibroblast IMR-90 was obtained from Coriell and cultured in Advanced DMEM/F-12 supplemented with 10% FBS and PSA.

Plasmids consisting of a U6 promoter driven chimeric guide RNA (sgRNA) and a ubiquitous promoter driven Cas9 were obtained from Addgene, as described by Cong L et al. (2013) Multiplex Genome Engineering Using CRISPR/Cas Systems. Science 339:819-823. An EGFP marker fused after the Cas9 protein allowed selection of Cas9-positive cells (FIG. 2). We adapted a modified chimeric guide RNA design for more efficient Pol-III transcription and more stable stem-loop structure (Chen B et al. (2013) Dynamic Imaging of Genomic Loci in Living Human Cells by an Optimized CRISPR/Cas System. Cell 155:1479-1491).

FIGS. 2-4 represent EBV-targeting CRISPR/Cas9 designs. FIG. 2 depicts a scheme of CRISPR/Cas plasmids, adapted from Cong L et al. (2013) Multiplex Genome Engineering Using CRISPR/Cas Systems. Science 339:819-823. FIG. 3 shows a graph of the effect of oriP on transfection efficiency in Raji cells. Both Cas9 and Cas9-oriP plasmids have a scrambled guide RNA. FIG. 4 depicts a CRISPR guide RNA targets along the EBV reference genome. Green, red and blue represent three different target sequence categories.

pX458 was obtained from Addgene, Inc., a modified CMV promoter with a synthetic intron (pmax) was PCR amplified from Lonza control plasmid pmax-GFP. A modified guide RNA sgRNA(F+E) was ordered from IDT. EBV replication origin oriP was PCR amplified from B95-8 transformed lymphoblastoid cell line GM12891. Standard cloning protocols were used to clone pmax, sgRNA(F+E) and oriP to pX458, to replace the original CAG promoter, sgRNA and fl origin. EBV sgRNA was designed based on the B95-8 reference, and DNA oligos were ordered from IDT. The original sgRNA place holder in pX458 serves as the negative control.

Lymphocytes are known for being resistant to lipofection, and therefore nucleofection was used for DNA delivery into Raji cells. The Lonza pmax promoter was chosen to drive Cas9 expression as it offered strong expression within Raji cells. The Lonza Nucleofector II was used for DNA delivery. 5 million Raji or DG-75 cells were transfected with 5 ug plasmids in each 100-ul reaction. Cell line Kit V and program M-013 were used following Lonza recommendation. For IMR-90, 1 million cells were transfected with 5 ug plasmids in 100 ul Solution V, with program T-030 or X-005. 24 hours after nucleofection, obvious EGFP signals were observed from a small proportion of cells through fluorescent microscopy. The EGFP-positive cell population decreased dramatically after that, however, and we measured <10% transfection efficiency 48 hours after nucleofection (FIG. 3). This transfection efficiency decrease was attributed to the plasmid dilution with cell division. To actively maintain the plasmid level within the cells, the CRISPR plasmid was redesigned to include the EBV origin of replication sequence, oriP. With active plasmid replication inside the cells, the transfection efficiency rose to >60% (FIG. 3).

To design guide RNA targeting the EBV genome, the EBV reference genome from strain B95-8 was relied upon. Six regions were targeted with seven guide RNA designs for different genome editing purposes.

Additional information such as primer design is shown in Wang and Quake, 2014, RNA-guided endonuclease provides a therapeutic strategy to cure latent herpesviridae infection, PNAS 111(36):13157-13162 and in the Supporting Information to that article published online at the PNAS website, and the contents of both of those documents are incorporated by reference for all purposes.

EBNA1 is crucial for many EBV functions including gene regulation and latent genome replication. Guide RNA sgEBV4 and sgEBV5 were targeted to both ends of the EBNA1 coding region in order to excise this whole region of the genome. Guide RNAs sgEBV1, 2 and 6 fall in repeat regions, so that the success rate of at least one CRISPR cut is multiplied. These “structural” targets enable systematic digestion of the EBV genome into smaller pieces. EBNA3C and LMP1 are essential for cell transformation, and guide RNAs sgEBV3 and sgEBV7 were designed to target the 5′ exons of these two proteins respectively.

### EBV Genome Editing.

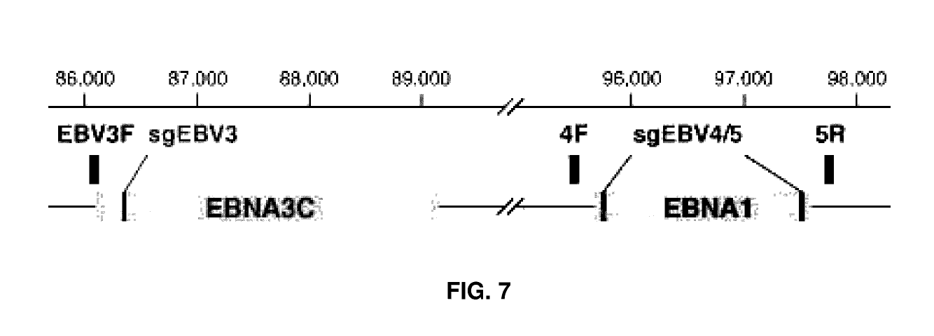

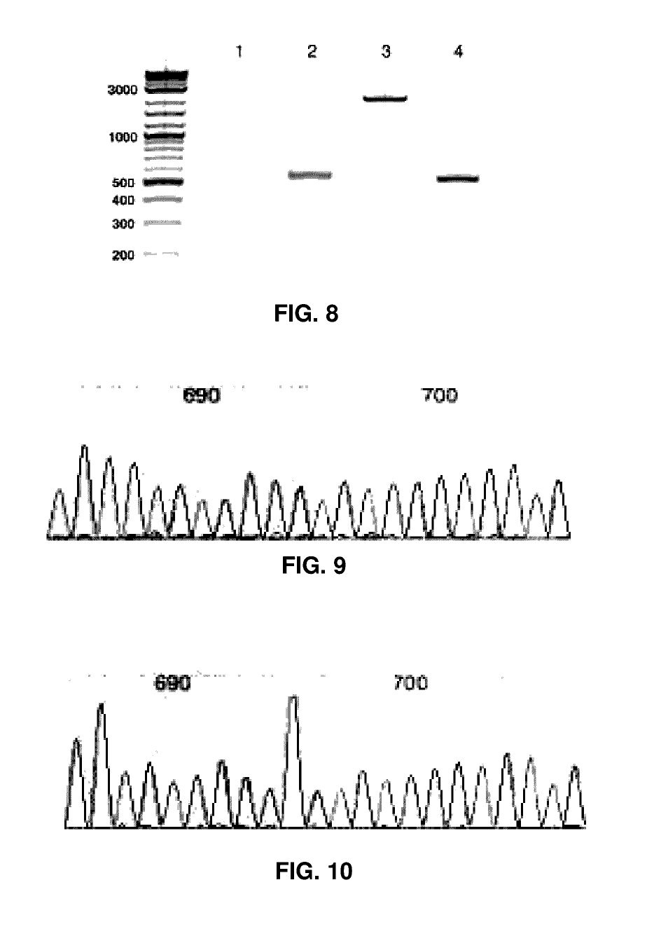

The double-strand DNA breaks generated by CRISPR are repaired with small deletions. FIGS. 5-9 represent CRISPR/Cas9 induced large deletions. FIG. 5 shows the genome context around guide RNA sgEBV2 and PCR primer locations. FIG. 6 shows the large deletion induced by sgEBV2. Lane 1-3 are before, 5 days after, and 7 days after sgEBV2 treatment, respectively. FIG. 7 shows the genome context around guide RNA sgEBV3/4/5 and PCR primer locations. FIG. 8 shows the large deletions induced by sgEBV3/5 and sgEBV4/5. Lane 1 and 2 are 3F/5R PCR amplicons before and 8 days after sgEBV3/5 treatment. Lane 3 and 4 are 4F/5R PCR amplicons before and 8 days after sgEBV4/5 treatment. FIGS. 9 and 10 show that Sanger sequencing confirmed genome cleavage and repair ligation 8 days after sgEBV3/5 (FIG. 9) and sgEBV4/5 (FIG. 10) treatment. Areas 690 and 700 (FIG. 9) and areas 690 and 700 (FIG. 10) indicate the two ends before repair ligation.

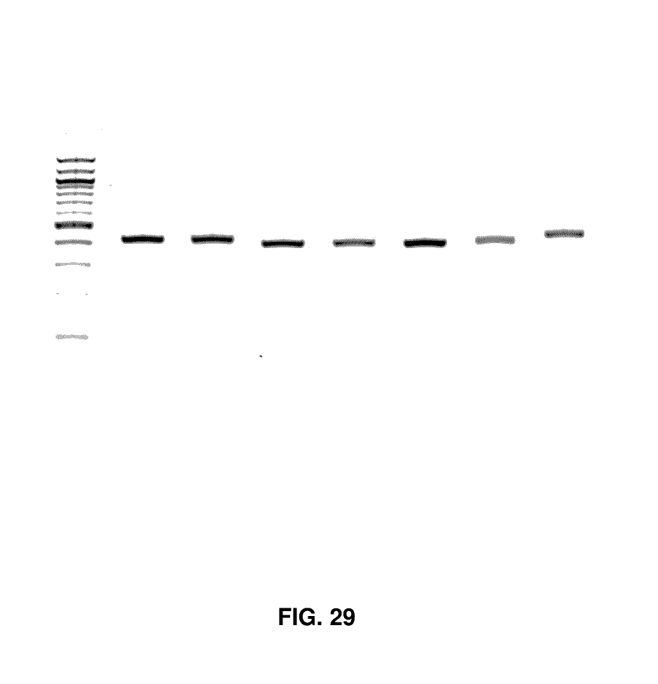

These deletions disrupt the protein coding and hence create knockout effects. SURVEYOR assays confirmed efficient editing of individual sites (FIG. 29). Beyond the independent small deletions induced by each guide RNA, large deletions between targeting sites can systematically destroy the EBV genome. Guide RNA sgEBV2 targets a region with twelve 125-bp repeat units (see FIG. 5). PCR amplicon of the whole repeat region gave a ˜1.8-kb band (see FIG. 6). After 5 or 7 days of sgEBV2 transfection, we obtained ˜0.4-kb bands from the same PCR amplification (FIG. 6). The ˜1.4-kb deletion is the expected product of repair ligation between cuts in the first and the last repeat unit (FIG. 5).

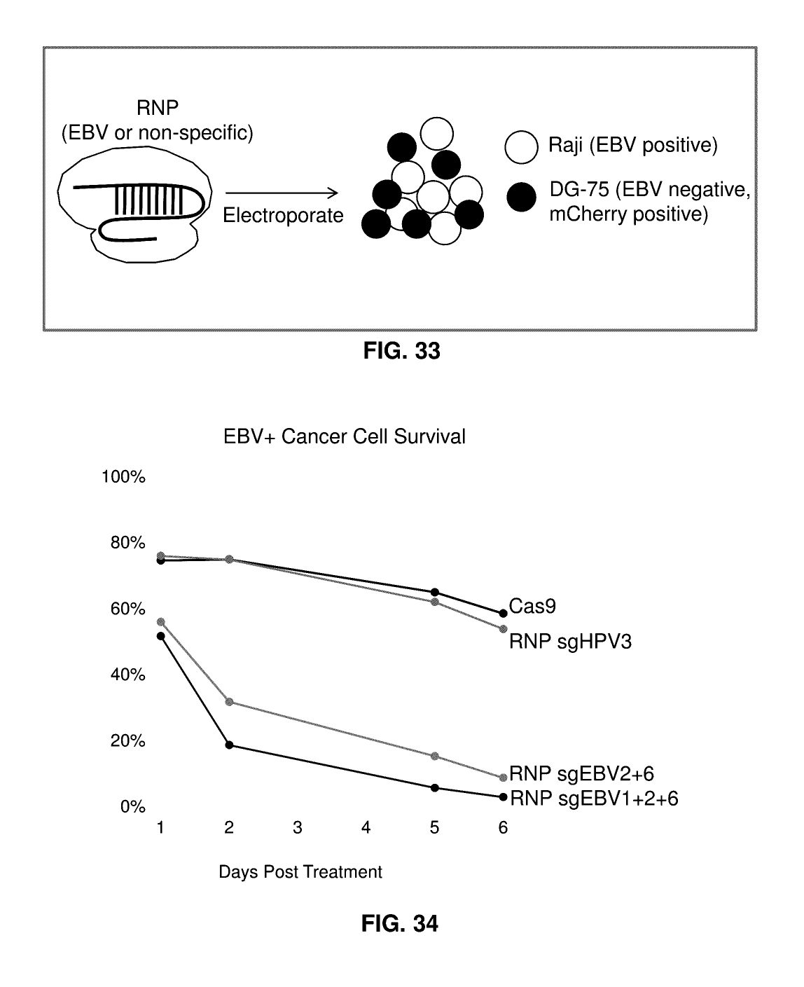

DNA sequences flanking sgRNA targets were PCR amplified with Phusion DNA polymerase (FIG. 33). SURVEYOR assays were performed following manufacturer's instruction. DNA amplicons with large deletions were TOPO cloned and single colonies were used for Sanger sequencing. EBV load was measured with Taqman digital PCR on Fluidigm BioMark. A Taqman assay targeting a conserved human locus was used for human DNA normalization. 1 ng of single-cell whole-genome amplification products from Fluidigm Cl were used for EBV quantitative PCR.

It is possible to delete regions between unique targets (FIG. 7). Six days after sgEBV4-5 transfection, PCR amplification of the whole flanking region (with primers EBV4F and 5R) returned a shorter amplicon, together with a much fainter band of the expected 2 kb (FIG. 8). Sanger sequencing of amplicon clones confirmed the direct connection of the two expected cutting sites (FIG. 10). A similar experiment with sgEBV3-5 also returned an even larger deletion, from EBNA3C to EBNA1 (FIGS. 8-9).

Cell Proliferation Arrest with EBV Genome Destruction.

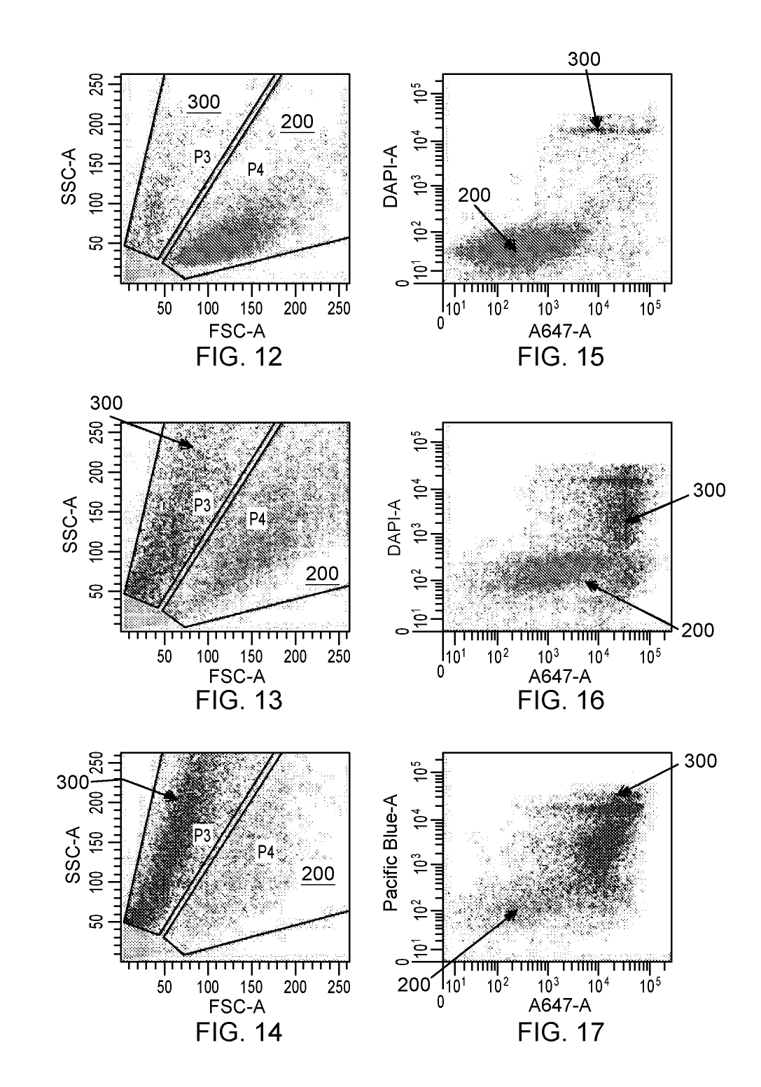

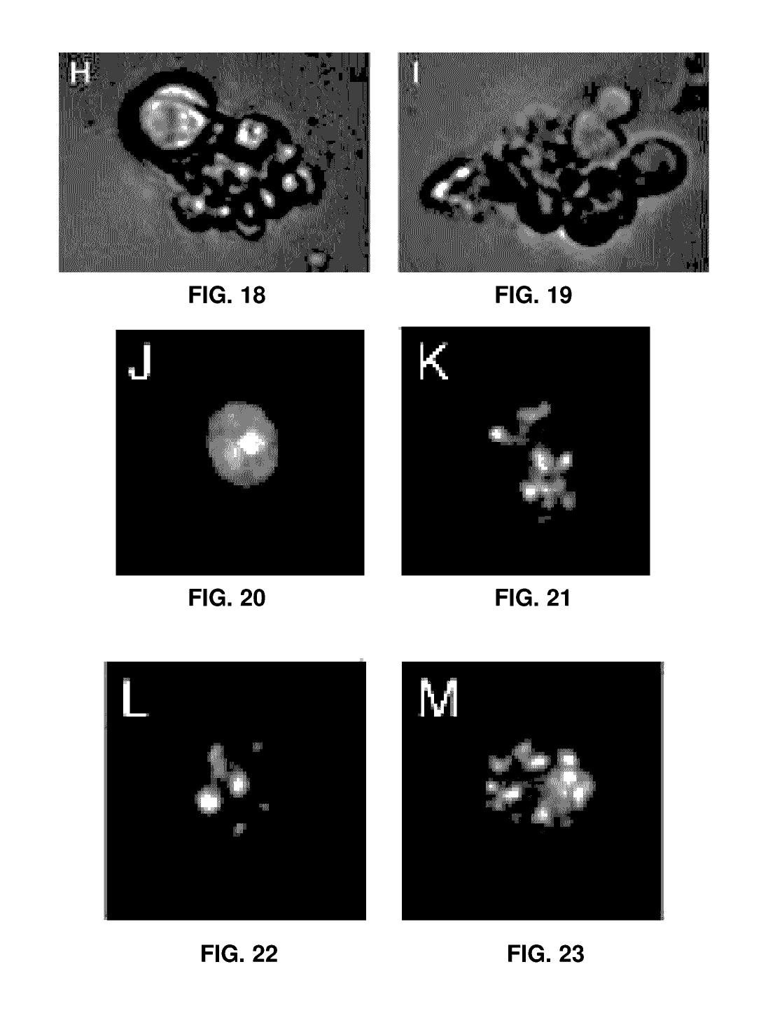

Two days after CRISPR transfection, EGFP-positive cells were flow sorted for further culture and counted the live cells daily. FIGS. 11-23 represent cell proliferation arrest with EBV genome destruction. FIG. 11 shows cell proliferation curves after different CRISPR treatments. Five independent sgEBV1-7 treatments are shown here. FIGS. 12-17 show flow cytometry scattering signals before (FIG. 12), 5 days after (FIG. 13) and 8 days after (FIG. 14) sgEBV1-7 treatments. FIG. 15-17 show Annexin V Alexa647 and DAPI staining results before (FIG. 15), 5 days after (FIG. 16) and 8 days after (FIG. 17) sgEBV1-7 treatments. Regions 300 and 200 correspond to subpopulation P3 and P4 in (FIGS. 12-14). FIGS. 18 and 19 show microscopy revealed apoptotic cell morphology after sgEBV1-7 treatment. FIGS. 20-23 show nuclear morphology before (FIG. 20) and after (FIGS. 21-23) sgEBV1-7 treatment.

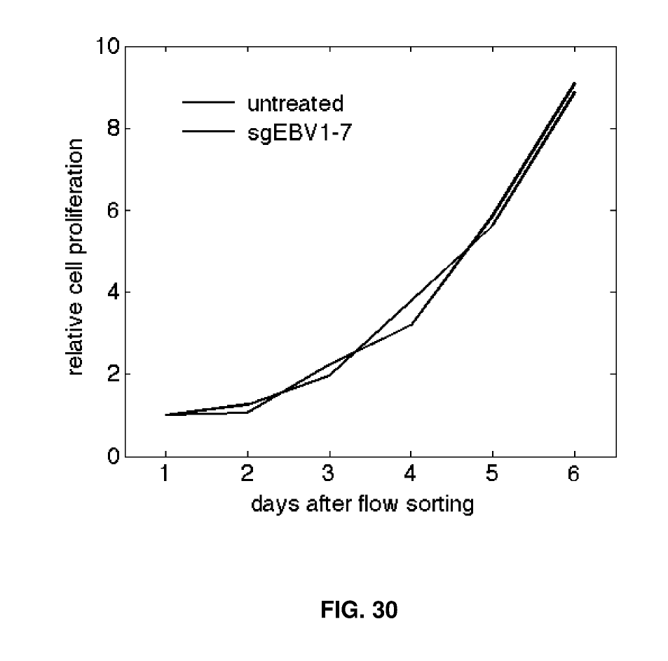

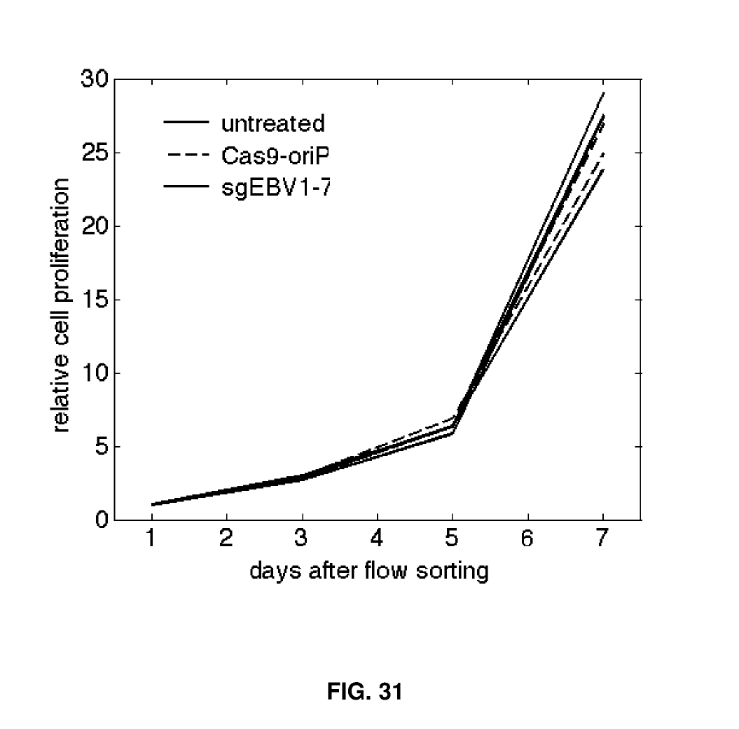

As expected, cells treated with Cas9 plasmids which lacked oriP or sgEBV lost EGFP expression within a few days and proliferated with a rate similar rate to the untreated control group (FIG. 11). Plasmids with Cas9-oriP and a scrambled guide RNA maintained EGFP expression after 8 days, but did not reduce the cell proliferation rate. Treatment with the mixed cocktail sgEBV1-7 resulted in no measurable cell proliferation and the total cell count either remained constant or decreased (FIG. 11). Flow cytometry scattering signals clearly revealed alterations in the cell morphology after sgEBV1-7 treatment, as the majority of the cells shrank in size with increasing granulation (FIG. 12-14, population P4 to P3 shift). Cells in population P3 also demonstrated compromised membrane permeability by DAPI staining (FIG. 15-17). To rule out the possibility of CRISPR cytotoxicity, especially with multiple guide RNAs, the same treatment was performed on two other samples: the EBV-negative Burkitt's lymphoma cell line DG-75 (FIG. 30) and primary human lung fibroblast IMR90 (FIG. 31). Eight and nine days after transfection the cell proliferation rates did not change from the untreated control groups, suggesting neglectable cytotoxicity.

Previous studies have attributed the EBV tumorigenic ability to its interruption of cell apoptosis (Ruf 1K et al. (1999) Epstein-Barr Virus Regulates c-MYC, Apoptosis, and Tumorigenicity in Burkitt Lymphoma. Molecular and Cellular Biology 19:1651-1660). Suppressing EBV activities may therefore restore the apoptosis process, which could explain the cell death observed in our experiment. Annexin V staining revealed a distinct subpopulation of cells with intact cell membrane but exposed phosphatidylserine, suggesting cell death through apoptosis (FIG. 15-17). Bright field microscopy showed obvious apoptotic cell morphology (FIG. 18-19) and fluorescent staining demonstrated drastic DNA fragmentation (FIG. 20-23). Altogether this evidence suggests restoration of the normal cell apoptosis pathway after EBV genome destruction.

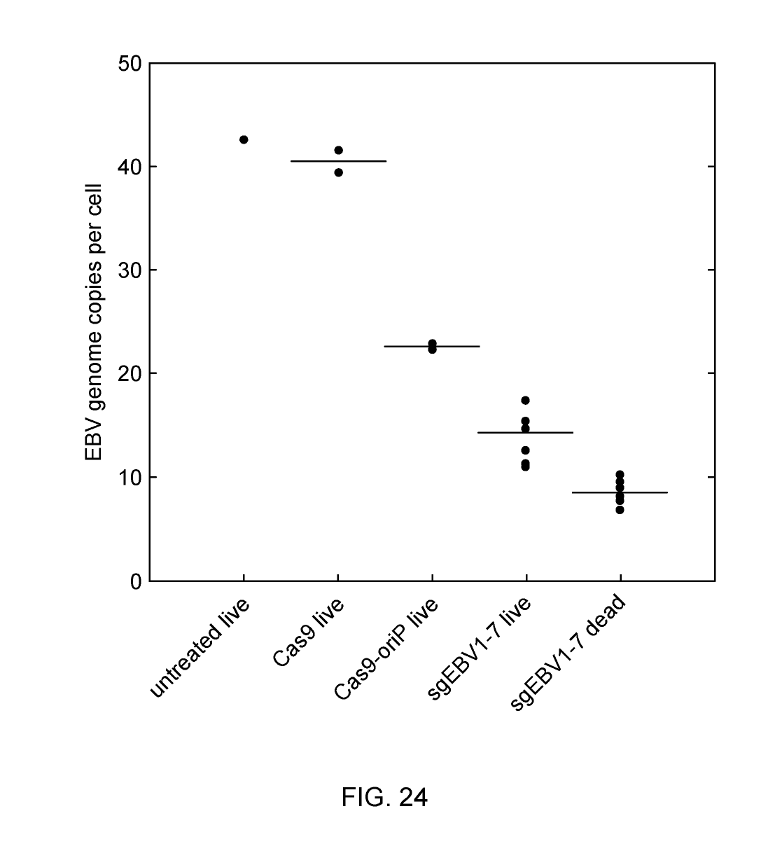

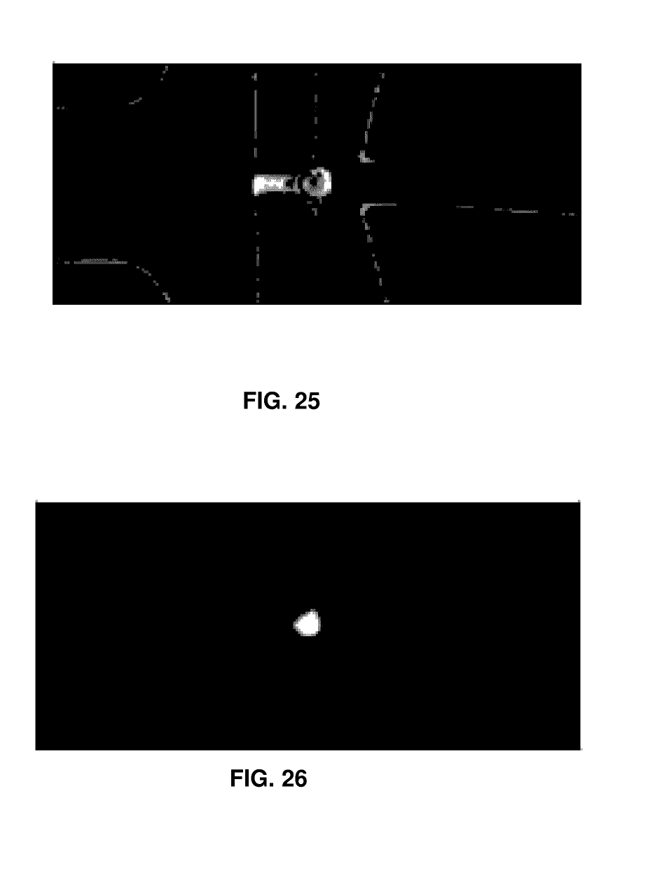

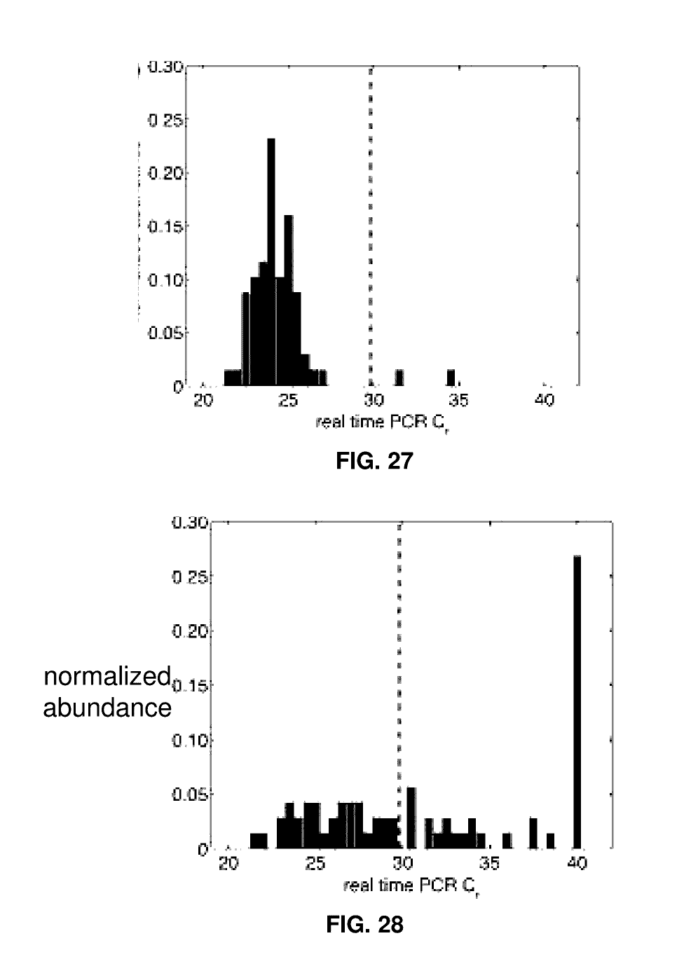

FIGS. 24-28 represent EBV load quantitation after CRISPR treatment. FIG. 24 shows EBV load after different CRISPR treatments by digital PCR. Cas9 and Cas9-oriP had two replicates, and sgEBV1-7 had 5 replicates. FIGS. 25 and 26 show microscopy of captured single cells for whole-genome amplification. FIG. 27 shows a histogram of EBV quantitative PCR Ct values from single cells before treatment. FIG. 28 shows a histogram of EBV quantitative PCR Ct values from single live cells 7 days after sgEBV1-7 treatment. The dash lines in (FIG. 27) and (FIG. 28) represent Ct values of one EBV genome per cell.

Complete Clearance Of EBV In A Subpopulation. To study the potential connection between cell proliferation arrest and EBV genome editing, the EBV load was quantified in different samples with digital PCR targeting EBNA1. Another Taqman assay targeting a conserved human somatic locus served as the internal control for human DNA normalization. On average, each untreated Raji cell has 42 copies of EBV genome (FIG. 24). Cells treated with a Cas9 plasmid that lacked oriP or sgEBV did not have an obvious difference in EBV load difference from the untreated control. Cells treated with a Cas9-plasmid with oriP but no sgEBV had an EBV load that was reduced by ˜50%. In conjunction with the prior observation that cells from this experiment did not show any difference in proliferation rate, we interpret this as likely due to competition for EBNA1 binding during plasmid replication. The addition of the guide RNA cocktail sgEBV1-7 to the transfection dramatically reduced the EBV load. Both the live and dead cells have >60% EBV decrease comparing to the untreated control.

Although seven guide RNAs were provided at the same molar ratio, the plasmid transfection and replication process is likely quite stochastic. Some cells will inevitably receive different subsets or mixtures of the guide RNA cocktail, which might affect the treatment efficiency. To control for such effects, the EBV load was measured at the single cell level by employing single-cell whole-genome amplification with an automated microfluidic system. Freshly cultured Raji cells were loaded onto the microfluidic chip and captured 81 single cells (FIG. 25). For the sgEBV1-7 treated cells, the live cells were flow sorted eight days after transfection and captured 91 single cells (FIG. 26). Following manufacturer's instruction, ˜150 ng amplified DNA was obtained from each single cell reaction chamber. For quality control purposes we performed 4-loci human somatic DNA quantitative PCR on each single cell amplification product (Wang J, Fan H C, Behr B, Quake S R (2012) Genome-wide single-cell analysis of recombination activity and de novo mutation rates in human sperm. Cell 150:402-412) and required positive amplification from at least one locus. 69 untreated single-cell products passed the quality control and displayed a log-normal distribution of EBV load (FIG. 27) with almost every cell displaying significant amounts of EBV genomic DNA. We calibrated the quantitative PCR assay with a subclone of Namalwa Burkitt's lymphoma cells, which contain a single integrated EBV genome. The single-copy EBV measurements gave a Ct of 29.8, which enabled us to determine that the mean Ct of the 69 Raji single cell samples corresponded to 42 EBV copies per cells, in concordance with the bulk digital PCR measurement. For the sgEBV1-7 treated sample, 71 single-cell products passed the quality control and the EBV load distribution was dramatically wider (FIG. 28). While 22 cells had the same EBV load as the untreated cells, 19 cells had no detectable EBV and the remaining 30 cells displayed dramatic EBV load decrease from the untreated sample.

FIG. 29 represents SURVEYOR assay of EBV CRISPR. Lane 1: NEB 100 bp ladder; Lane 2: sgEBV1 control; Lane 3: sgEBV1; Lane 4: sgEBV5 control; Lane 5: sgEBV5; Lane 6: sgEBV7 control; Lane 7: sgEBV7; Lane 8: sgEBV4. FIG. 30 represents CRISPR cytotoxicity test with EBV-negative Burkitt's lymphoma DG-75. FIG. 31 represents CRISPR cytotoxicity test with primary human lung fibroblast IMR-90.

Essential Targets For EBV Treatment. The seven guide RNAs in our CRISPR cocktail target three different categories of sequences which are important for EBV genome structure, cell transformation, and infection latency, respectively. To understand the most essential targets for effective EBV treatment, we transfected Raji cells with subsets of guide RNAs. Although sgEBV4/5 reduced the EBV genome by 85%, they could not suppress cell proliferation as effectively as the full cocktail (FIG. 11). Guide RNAs targeting the structural sequences (sgEBV1/2/6) could stop cell proliferation completely, despite not eliminating the full EBV load (26% decrease). Given the high efficiency of genome editing and the proliferation arrest (FIG. 2), we suspect that the residual EBV genome signature in sgEBV1/2/6 was not due to intact genomes but to free-floating DNA that has been digested out of the EBV genome, i.e. as a false positive. We conclude that systematic destruction of EBV genome structure appears to be more effective than targeting specific key proteins for EBV treatment.

### Example 2

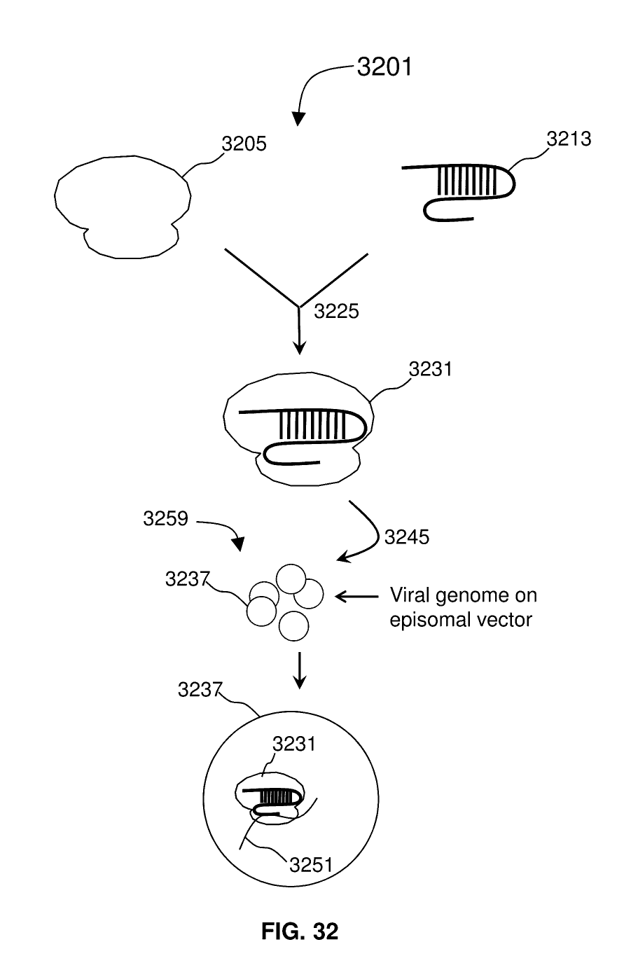

FIG. 32 shows a method 3201 for treating a cell 3237 to remove foreign nucleic acid such as a viral nucleic acid 3251. The method 3201 may be used to a support a hematopoietic stem cell transplant (HSCT) procedure, or the method 3201 may be used in vitro in research and development to remove foreign nucleic acid from subject cells such as cells from a human.

The method 3201 includes the steps of: forming 3225 a ribonucleoprotein (RNP) 3231 that includes a nuclease 3205 and an RNA 3213; obtaining a cell 3237 from a donor; delivering 3245 (preferably in vitro) the RNP 3231 to the cell 3237; and cleaving viral nucleic acid 3251 within the cell 3237 with the RNP 3231. The method 3201 may include providing the cell 3237 for transplantation into a patient.

The delivering 3245 may include electroporation, or the RNP may be packaged in a liposome for the delivering 3245. In some embodiments, the viral nucleic acid 3251 will exist as an episomal viral genome, i.e., an episome or episomal vector, of a virus. The RNA 3213 has a portion that is substantially complementary to a target within a viral nucleic acid 3251 and preferably not substantially complementary to any location on a human genome. In the preferred embodiments, the virus is a herpes family virus such as one selected from the group consisting of HSV-1, HSV-2, Varicella zoster virus, Epstein-Barr virus, and Cytomegalovirus. The virus may be in a latent stage in the cell.

In a preferred embodiment, the nuclease 3205 is a Crisper-associated protein such as, preferably, Cas9. The RNA 3213 may be a single guide RNA (sgRNA) (providing the functionality of crRNA and tracrRNA). In the preferred embodiment, the nuclease 3205 and the RNA 3213 are delivered to the cell as the RNP 3231.

In some embodiments, the patient is a pre-determined person who has a human leukocyte antigen (HLA) type matched to the donor. The patient may be the donor. The cell 3237 may be a hematopoietic stem cell (e.g., obtained from the donor's bone marrow or peripheral blood). In preferred embodiments, the cell 3237 has the viral nucleic acid 3251 therein, and the method further comprises cleaving the viral nucleic acid using the nuclease.

The method 3201 may include delivering the RNP 3231 to a plurality of cells 3259 from the donor, culturing the plurality of cells, and selecting the cell 3237 from among the plurality of cells 3259 based on successful cleavage of the viral nucleic acid. Selecting the cell may include using a fluorescent marker delivered with the nuclease.

In some embodiments, it may be found that RNP is preferable (e.g., to plasmid DNA) for clinical applications, particularly for parenteral delivery. RNP is the active pre-formed drug which offers advantages to DNA (AAV) or mRNA. No need to transcribe, translate, or assemble drug components within cell. Delivery of RNP 3231 may offer improved drug properties, e.g. earlier onset activity and controlled clearance (toxicity).

EBV-specific CRISPR/Cas9 RNP specifically kills EBV+B lymphoma cancer cells.

FIG. 33 diagrams an experimental design to show that EBV-specific CRISPR/Cas9 RNP specifically kills EBV+B lymphoma cancer cells. The Raji cells are EBV positive. Raji cells are a continuous human cell line of hematopoetic origin. The DG-75 cells are an EBV-negative B lymphocyte cell line available from American Type Culture Collection (Manassas, Va.). The DG-75 exhibits an mCherry fluorescent marker. Since the EBV negative cells contain a fluorescent marker, successful cleavage events can be identified.

FIG. 34 shows EBV+cancer cell survival for 6 days post-treatment. Those EBV+cells that received the RNP 3231 with guide RNAs substantially complementary to Epstein-Barr viral nucleic acid 3251 exhibited <10% survival rate, compared to about 60-70% in controls.

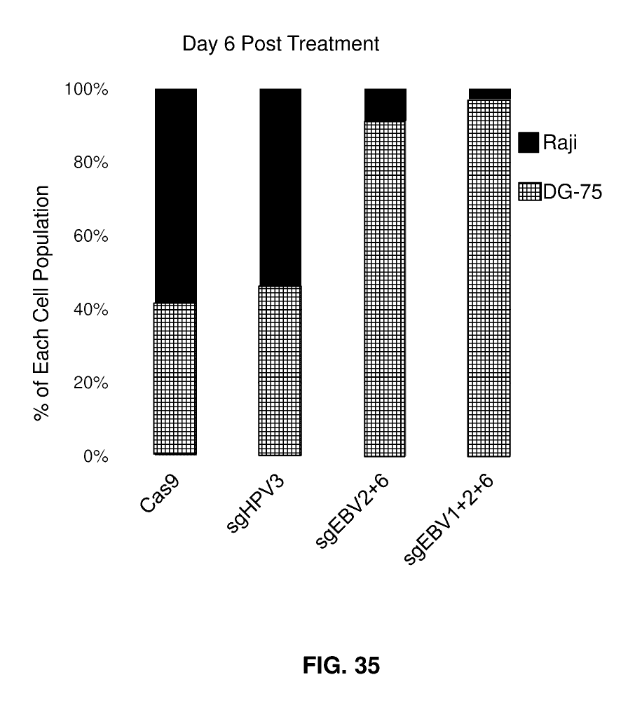

FIG. 35 shows the percent of each cell population at day 6 post-treatment for Cas9, sgHPV3, sgEBV2+6, and sgEBV1+2+6. This snapshot at day 6 shows that the DG-75 treated with the RNP 3231 with guide RNAs substantially complementary to Epstein-Barr viral nucleic acid 3251 dominated the cultures over the Raji cells.

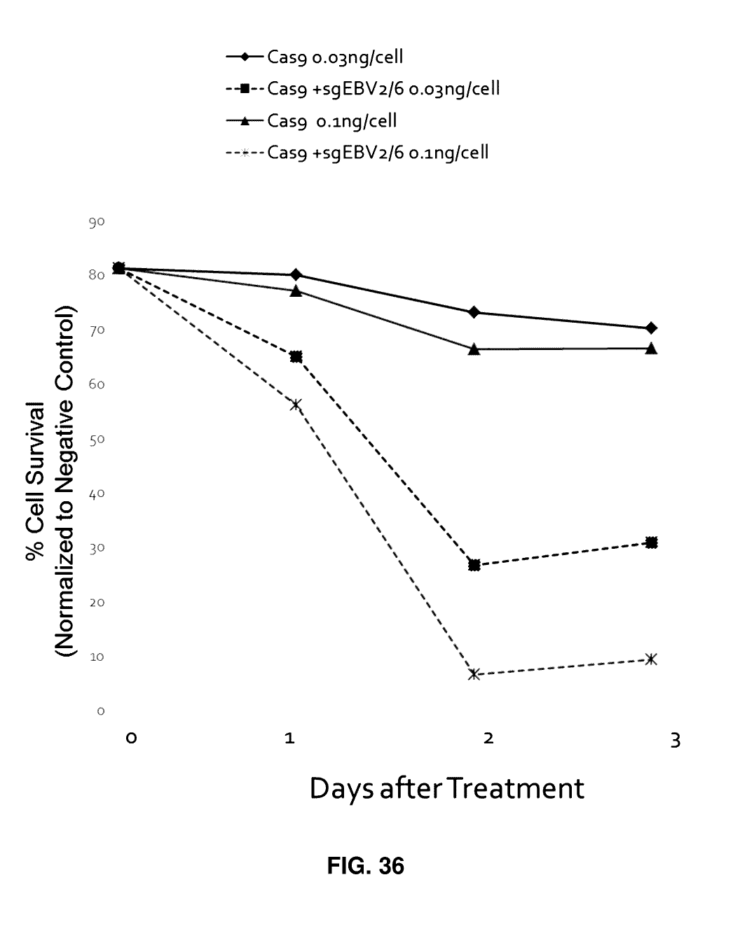

FIG. 36 shows the percent cell survivial (normalized to a negative control) for 3 days after treatment for Cas9 (at 0.03 & 0.1 ng/cell) as well as for Cas9 with sgEBV2/6 (at the same doses).

What is claimed is:

1. A method for generating a viral-free cell, the method comprising the steps of:
obtaining a cell from a donor;
delivering to the cell a nuclease that cleaves viral nucleic acid; and
providing the cell for transplantation into a patient.

2. The method of claim 1, wherein the patient is a pre-determined person who has a human leukocyte antigen (HLA) type matched to the donor.

3. The method of claim 1, wherein the patient is the donor.

4. The method of claim 1, wherein the cell is a hematopoietic stem cell.

5. The method of claim 1, wherein the cell is obtained from the donor's bone marrow or peripheral blood.

6. The method of claim 1, wherein the nuclease includes one selected from the group consisting of a zinc finger nuclease, a transcription activator-like effector nuclease, and a meganuclease.

7. The method of claim 1, wherein the nuclease is a Cas9 endonuclease.

8. The method of claim 7, further comprising delivering to the cell a guide RNA that targets the Cas9 endonuclease to a portion of the viral nucleic acid.

9. The method of claim 8, wherein the nuclease and the guide RNA are delivered to the cell as a ribonucleoprotein.

10. The method of claim 9, wherein the cell is infected by a virus and has the viral nucleic acid therein, and the method further comprises cleaving the viral nucleic acid using the nuclease.

11. The method of claim 10, further comprising delivering the nuclease to a plurality of cells from the donor, culturing the plurality of cells, and selecting the cell from among the plurality based on successful cleavage of the viral nucleic acid.

12. The method of claim 11, wherein selecting the cell comprises using a fluorescent marker delivered with the nuclease.

13. The method of claim 9, wherein the virus is a herpes family virus.

14. The method of claim 1, wherein the virus is in a latent stage in the cell.

15. The method of claim 1, wherein the delivering step comprises delivering the nuclease in a viral vector.

16. The method of claim 15, wherein the viral vector is selected from the group consisting of retrovirus, lentivirus, adenovirus, herpesvirus, poxvirus, alphavirus, vaccinia virus and adeno-associated viruses.

17. The method of claim 1, wherein the delivering step comprises delivering the nuclease in a vector that includes one selected from the group consisting of a plasmid, a nanoparticle, a cationic lipid, a cationic polymer, metallic nanoparticle, a nanorod, a liposome, a cell-penetrating peptide, a liposphere, and polyethyleneglycol (PEG).

18. The method of claim 10, wherein cleaving comprises causing one or more double strand breaks in the viral genome.

19. The method of claim 10, wherein cleaving comprises causing an insertion in the viral genome.

20. The method of claim 1, wherein the cell is infected by a virus and has the viral nucleic acid therein, and the method further comprises cleaving the viral nucleic acid using the nuclease.

21. The method of claim 20, further comprising delivering the nuclease to a plurality of cells from the donor, culturing the plurality of cells, and selecting the cell from among the plurality based on successful cleavage of the viral nucleic acid.

22. The method of claim 21, wherein selecting the cell comprises using a fluorescent marker delivered with the nuclease.
